(19) United States
(12) Patent Application Publication
(10) Pub. No.: US 2024/0102177 A1
(43) Pub. Date: Mar. 28, 2024

(54) ELECTROCHEMICAL COFACTOR REGENERATION USING EARTH ABUNDANT ELECTRODES FOR BIOCATALYTIC APPLICATIONS

(71) Applicant: King Adbullah University of Science and Technology, Thuwal (SA)

(72) Inventors: Jeremy Adrian BAU, Thuwal (SA); Magnus RUEPING, Thuwal (SA); Dominik RENN, Thuwal (SA)

(21) Appl. No.: 18/275,892

(22) PCT Filed: Feb. 4, 2022
(86) PCT No.: PCT/IB2022/051003

§ 371 Date: Aug. 4, 2023

Related U.S. Application Data
(60) Provisional application No. 63/145,656, filed on Feb. 4, 2021.

(51) Int. Cl. C25B 1/01; C12P 3/00; C12P 7/22; C25B 3/25; C25B 9/19; C25B 11/075

(52) U.S. Cl. CPC C25B 1/01; C12P 3/00; C12P 7/22; C25B 3/25; C25B 9/19; C25B 11/075; C12Y 101/01001

(57) ABSTRACT

Embodiments of the present disclosure describe methods and systems using a hydride-forming Group VI transition metal chalcogenide catalyst, such as MoSx, for selective electrocatalysis of enzyme cofactor regeneration. In particular, a method of electrochemical cofactor regeneration comprising: holding an electrode comprising a Group VI transition metal chalcogenide catalyst at a potential sufficient to form a metal hydride in an aqueous electrolyte solution; and contacting the electrode with an oxidized cofactor to reduce the cofactor, is provided. The reduced cofactor can be used by a cofactor-dependent oxidoreductase to convert a substrate to a desired product and subsequently regenerated.

### BACKGROUND

H₂ generated from water electrolysis powered by renewable energy is a strong candidate as an alternative energy vector and carbon-neutral reducing agent, but its mass implementation requires the development of earth-abundant catalysts for the H₂-evolution reaction (HER) and the O₂-evolution reaction, both of which remain dominated by precious metals. A successful zero-carbon transition also requires the replacement of traditional high temperature chemical processes with cleaner alternatives that function under facile conditions, such as via bio- or electrocatalysis. Hydride transfers are key to H₂ evolution.

The transfer of energy through hydrides is of particular significance to the field of biocatalysis, where one quarter of all known enzymes, known as oxidoreductases, are capable of catalyzing electron and hydride transfers in the presence of hydride-bearing co-factors. The vast majority of oxidoreductases (90%) rely on the biological energy carrier nicotinamide adenine dinucleotide (NAD) and its reduced, hydrogenated form (NADH) or their phosphorylated forms (NADP(H)) as co-factors. Electrochemical NAD(P)H regeneration has long been considered promising, but single electron transfers often result in the formation of biologically inactive NAD dimers or 1,2- and 1,6-dihydropyridine products. Electrochemical catalysts for cofactor regeneration, such as NAD(P)H regeneration, that are capable of avoiding single electron transfers and also direct hydride transfer are needed.

### SUMMARY

The present disclosure describes the use of a hydride-forming Group VI transition metal chalcogenide catalyst, such as MoSₓ, for economical and selective enzyme cofactor regeneration. This use is predicated on Applicant's discovery that Group VI transition metal chalcogenides form a hydride active species at cathodic potentials in aqueous solutions. The ability of Group VI transition metal chalcogenide electrocatalysts to form and transfer hydrides in exclusion of single electron transfers opens a cost-effective route for application in biocatalysis and a new paradigm for electrocatalyst design.

Accordingly, a first aspect of the present disclosure features a method electrochemical cofactor regeneration comprising holding an electrode including a Group VI transition metal chalcogenide catalyst at a potential sufficient to form a metal hydride in an aqueous electrolyte solution; contacting the electrode with an oxidized cofactor to reduce the cofactor. The oxidized cofactor can be selected from the group consisting of cofactor of NAD⁺, NADP⁺, FAD⁺ and FMN⁺ or a combination thereof. The potential can be held within the range of about −0.3V to −0.6 V. The Group VI transition metal chalcogenide catalyst has the formula MEₓ, where M is a Group VI transition metal, E is a non-metal element, and x is a number greater than 2. M can be selected from Cr, Mo, and W, E can be selected from the group of non-metal elements consisting of B, C, N, S, Se, Te, and P, or both M and E can be Cr, Mo, or W and B, C, N, S, Se, Te, or P, respectively. The Group VI transition metal chalcogenide catalyst can be selected from the group of MoSₓ, MoSeₓ, WSeₓ, and WSₓ, wherein x is a number greater than 2. The Group VI transition metal chalcogenide catalyst can be amorphous. The aqueous electrolyte solution can be configured to have an alkaline or neutral pH. The aqueous electrolyte solution can include least one of potassium phosphate, sodium phosphate and potassium perchlorate.

A second aspect of the present invention features a method of improving the rate of an oxidoreductase-catalyzed reaction, the method comprising reacting an oxidoreductase and a substrate thereof in the presence of an oxidoreductase cofactor, whereby the substrate is converted to a first product and the cofactor is oxidized; regenerating the oxidized cofactor according to the method of one or more embodiments of the first aspect with an electrode comprising a Group VI transition metal chalcogenide catalyst at a potential sufficient to form a metal hydride, wherein the rate of the reaction is improved compared to the rate of a corresponding oxidoreductase-catalyzed reaction performed without regenerating the oxidized cofactor. The oxidoreductase can be selected from the group consisting of nicotinamide-dependent oxidoreductases, NADH-dependent oxidoreductases, NADPH-dependent oxidoreductases, and FADH₂-dependent oxidoreductases. The oxidoreductase can be selected from the group consisting of alcohol dehydrogenases, aldehyde dehydrogenases, ene reductases, amino acid dehydrogenases, oxidoreductases of CH—NH groups, nitrate reductases, oxidoreductases acting on a sulfur group, dehydrogenases of diphenols, peroxidases, hydrogenases, oxygenases, monooxygenases, oxidoreductases of metal ions, oxidoreductases acting on CH or CH₂ groups, oxidoreductases of iron-sulfur proteins and of flavodoxin, reductive dehalogenases, and oxidoreductases reducing a C—O—C group. The oxidoreductase can be conjugated to the electrode. The oxidoreductase can be solubilized in the aqueous electrolyte solution. The electrode can be contained in a reaction vessel and the oxidoreductase can be separated from the electrode by a membrane. The oxidoreductase can be retained by a dialysis membrane or immobilized on the membrane. The method can further include a step of reacting a second enzyme and the first product, whereby the first product is converted to a second product. In some embodiments, the method includes reacting a third enzyme and the second product, whereby the second product is converted to a third product.

In a third aspect, the present disclosure features a system for an oxidoreductase-catalyzed reaction comprising a bioreactor including a reaction vessel; wherein the reaction vessel is configured to contain an electrode comprising a Group VI transition metal chalcogenide catalyst and an aqueous electrolyte solution; the reaction vessel further configured for regenerating a oxidoreductase cofactor according to a method of one or more embodiments of the first aspect; wherein the bioreactor is configured for reacting the oxidoreductase and a substrate thereof in the presence of the regenerated cofactor. The system can further include a membrane configured to separate the oxidoreductase and the electrode. The membrane can be dialysis membrane. The oxidoreductase can be immobilized on the membrane. The oxidoreductase can be conjugated to the electrode. The system can further include a counter electrode and a salt bridge. The salt bridge can include an ion exchange membrane.

### BRIEF DESCRIPTION OF DRAWINGS

The following written disclosure describes various embodiments. Illustrative examples are provided in the accompanying drawings, in which:

FIGS. 1A-C is a scheme of (A) the classic MoS₂ edge site-based HER mechanism in contrast to (B) the Mo³⁺ hydride mechanism discussed in this study. (C) Plausible reaction mechanism of how Mo³⁺ hydride in a-MoSₓ catalyzes the HER and NADH regeneration mechanisms, and is subsequently regenerated, according to one or more embodiments of the present disclosure.

FIG. 2 is an illustration of a system and method of using a Group VI transition metal chalcogenide catalyst (MoSx) for regenerating an enzymatic cofactor electrochemically, according to one or more embodiments of the present disclosure.

FIG. 3 is an illustration of a system for regenerating an enzymatic cofactor electrochemically using a Group VI transition metal chalcogenide electrocatalyst combined with a membrane-separated soluble enzyme, according to one or more embodiments of the present disclosure.

FIG. 4 is an illustration of a system for regenerating an enzymatic cofactor electrochemically using a Group VI transition metal chalcogenide electrocatalyst with an electrode-conjugated enzyme, according to one or more embodiments of the present disclosure

FIG. 5 is an illustration of a system for regenerating an enzymatic cofactor electrochemically using a Group VI transition metal chalcogenide electrocatalyst for a multi-enzyme biocatalytic cascade, according to one or more embodiments of the present disclosure.

FIGS. 6A-D describe Electron Paramagnetic Resonance (EPR) and electrochemical evidence for a Mo³⁺ hydride in a-MoSₓ. (A) EPR spectra of a trapped Mo³⁺ hydride. (B) Cyclic voltammogram of a-MoSₓ in 0.2 M Bu₄N PF₆/THF. Inset, EPR spectrum of a-MoSₓ reduced at −2.5 V vs. Ag/Ag⁺. Signal at 2.002 arises from the reduction of the electrolyte. (C) Correlation plot of Mo³⁺ peak size in 0.2 M Bu₄N PF₆/THF vs. HER activity as measured by η at −10 mA cm⁻² in 0.05 M H₂SO₄. (D) EPR spectra of anodically deposited a-MoSₓ after deposition and after conditioning by voltammetric sweep to the HER potential, according to one or more embodiments of the present disclosure.

FIGS. 7A-F shows example traces of a-MoSₓ scanned using (A, 10 cycles; B, 20 cycles, and C, 40 cycles) linear sweep voltammograms in 0.5 M H₂SO₄ (scanned at 1 mV s⁻¹) and (D, 10 cycles, D, 20 cycles, and F, 40 cycles) 0.2 M Bu₄N PF₆. The overpotentials from (a-c) and the peak areas in (d-f) are used to generate FIG. 6C.

FIG. 8 is a graphical representation of Easyspin simulations of EPR spectra for Mo³⁺ hydride and a-MoSₓ.

FIG. 9 shows TABLE 1 describing the EPR parameters of Mo³⁺ hydride according to one or more embodiments of the present disclosure, and literature reported values. ⁶Prior, C. et al. Dalton Trans. 45, 2399-2403 (2016); ⁷Baya, M. et al. Angew. Chem. Intl. Ed. 46, 429-432 (2007); ⁸Kinney, R. A. et al. Inorg. Chem. 49, 704-713 (2010); ⁹Tsai, Y.-C. et al. Organometallics 22, 2902-2913 (2003).

FIG. 10 show example organic sweeps in 0.2 M Bu₄N PF₆ (left) and linear sweep voltammograms in 0.05 M H₂SO₄ (right) of MoS₂ electrodes prepared by autoclave synthesis, according to one or more embodiments of the present disclosure.

FIG. 11 shows an XRD pattern of MoS₂ electrodes prepared by autoclave synthesis, according to one or more embodiments of the present disclosure.

FIGS. 12A-B show example organic sweeps in 0.2 M Bu₄N PF₆ (A) and linear sweep voltammograms in 0.05 M H₂SO₄ (B) of epitaxially-grown single-crystal MoS₂ films.

FIGS. 13A-B show electron micrographs of a-MoSₓ (A) and autoclave-prepared (hydrothermal) MoS₂ (B).

FIGS. 14A-D are X-ray photoelectron spectra of Mo3d and S2p for a-MoSₓ ((A) and (B), respectively) and autoclave-prepared (hydrothermal) MoS₂ ((C) and (D), respectively).

FIGS. 15A-C describe electrochemical and absorption characteristics of NMN reduction by a-MoSₓ. (A) Voltammograms of a-MoSₓ and GC in 0.5 M K₂CO₃ with and without NMN. (B) Linear sweep voltammograms of a-MoSₓ for the HER and NMN reduction. (C) UV-vis spectra of 5 mM NMN solution being reduced over time in 0.5 M K₂CO₃ solution (pH 10) by a-MoSₓ electrode (−10 mA cm⁻²). Inset, reduction of NMN by FTO. Dimer decomposition peak marked **.

FIGS. 16A-L describe reduction of NMN to 1,4-dihydropyridine derivative according to one or more embodiments of the present disclosure. (A) Expected structure of a mixed HD 1,4-dihydropyridine. (B)²H-NMR spectrum of the products of the D₂O reaction. (C,E)¹³C-NMR and ¹H-NMR spectra of the isolated products when the reaction is carried out in D₂O, respectively; (D,F)¹³C-NMR and ¹H-NMR spectra of the products in the H₂O reactions, respectively. Highlighted insets in (i) are the triplet peaks. A full spectrum is included in each inset; (G) shows the NMR spectrum of N-methylnicotinamide (NMN) starting material. (H) Full ²H NMR spectrum (reaction run in D₂O). (I) Full ¹³C NMR spectrum (reaction run in D₂O). Marked peaks correspond to splitting arising from CDH groups. (J) Full ¹H NMR spectrum (reaction run in D₂O). (K) Full 13C NMR spectrum (reaction run in H₂O). (L) Full ¹H NMR spectrum of reaction run in H₂O.

FIG. 17A depicts conversion of benzaldehyde to benzyl alcohol by S. cerevisiae ADH using a-MoSₓ as a catalyst for NAD reduction to NADH, according to one or more embodiments of the present disclosure.

FIG. 17B illustrates the ratio of NAD dimer to NADH formed during electrolysis by holding at −600 mV, according to one or more embodiments of the present disclosure, as assayed by UHPLC-MS. To facilitate UHPLC, 0.1 M ammonium acetate (pH 9) was used as the electrolyte.

FIG. 18A shows TABLE 2, describing the yield of biocatalytic conversion of benzaldehyde to benzyl alcohol with different substrates, including an electrocatalyst according to one or more embodiments of the present invention.

FIG. 18B shows TABLE 3, describing the yield of electrochemical conversion of NAD to NADH with different substrates, including an electrocatalyst according to one or more embodiments of the present invention.

FIG. 19A graphically depicts a time course of NADH regeneration and biocatalytic benzyl alcohol synthesis using a-MoSₓ in 0.1 M CHES/0.1 M K₂SO₄ (pH 9), according to one or more embodiments of the present disclosure.

FIG. 19B illustrates NADH regeneration and biocatalytic benzyl alcohol synthesis after 3 hours using a-MoSₓ, hydrothermal MoS₂ (h-MoS₂), defect-free MoS₂, Ti, and GC, according to one or more embodiments of the present disclosure. NADH regeneration was carried out as in (FIG. 18B), in the absence of enzyme and benzaldehyde.

FIG. 20 shows ultrahigh performance liquid chromatography (UHPLC) and mass spectra (experimental and theoretical) of NAD dimer in NAD reduced by a-MoSₓ in 0.1 M ammonium acetate (pH 9).

FIG. 21 shows UHPLC of NAD dimer-DBA ion pair in NAD reduced by a-MoSₓ in 0.1 M ammonium acetate (pH 9).

FIG. 22 shows UHPLC and mass spectra (experimental and theoretical) of NADH-DBA ion pair in NAD reduced by a-MoSₓ in 0.1 M ammonium acetate (pH 9).

FIG. 23 shows UHPLC and mass spectra (experimental and theoretical) of NADH in NAD reduced by a-MoSₓ in 0.1 M ammonium acetate (pH 9).

FIG. 24 shows UHPLC and experimental mass spectrum of NAD dimer in NAD reduced by a-MoSₓ in 0.1 M ammonium acetate (pH 9).

FIG. 25 shows UHPLC and experimental mass spectrum of NAD dimer-DBA ion pair in NAD reduced by a-MoSₓ in 0.1 M ammonium acetate (pH 9). The MS filter values were the same used in FIG. 22.

FIGS. 26A-C show (A) N1s and (B) S2p XPS of 6-maleimidohexanoic acid-functionalized a-MoSₓ electrodes. N1s at 400 eV corresponds to the N of maleimide. (C) shows TABLE 4 describing quantification of N to S.

FIGS. 27A-B are polarization curves of ultrathin a-MoSₓ electrodes in neutral (A) and acidic (B) electrolytes, prepared by 5 electrodeposition cycles, compared to the same electrodes functionalized with maleimide derivative. Inset. Average overpotentials (at −10 mA cm⁻²) of ultrathin a-MoSₓ electrodes compared to those functionalized with maleimide derivative.

FIG. 28 is a diagram of an electrochemical EPR setup, according to one or more embodiments of the present disclosure.

FIG. 29 is a photograph of a-MoSₓ electrode prepared by electrodeposition for use as a cofactor reduction catalyst, according to one or more embodiments of the present disclosure.

FIG. 30 shows TABLE 5 describing the reduction potential of electrochemical reduction half-reactions of the three major oxidoreductase cofactors and the formation potential (estimated) for a Group VI metal hydride, where “M” is the Group VI metal, according to one or more embodiments of the present disclosure.

FIG. 31 is a GC-MS spectra of the conversion of benzaldehyde to benzyl alcohol after 1 hour using an immobilized ADH on the surface of a Group VI transition metal chalcogenide electrocatalyst, according to one or more embodiments of the present disclosure.

### DETAILED DESCRIPTION

The present disclosure describes methods and systems using a hydride-forming Group VI transition metal chalcogenide catalyst, such as MoSₓ for economical and selective electrocatalysis of cofactor regeneration. The embodiments of the present disclosure are predicated on Applicant's discovery that Group VI transition metal chalcogenides form a hydride active species at cathodic potentials in aqueous solutions. The ability of Group VI transition metal chalcogenide electrocatalysts to form and transfer hydrides in exclusion of single electron transfers opens a cost-effective route for application in biocatalysis and a new paradigm for electrocatalyst design.

In one embodiment of the present disclosure, a method of electrochemical cofactor regeneration includes holding an electrode comprising a Group VI transition metal chalcogenide at a potential sufficient to form a metal hydride in an aqueous electrolyte solution; and contacting the electrode with an oxidized cofactor to reduce the cofactor. The Group VI transition metal chalcogenide catalysts of the present disclosure are semiconducting and have electrochemical activity for the specific reduction biological redox cofactors, especially but not limited to NAD⁺, NADP⁺, and FAD(H)⁺, to their reduced counterparts, namely NADH, NADPH, and FADH₂, respectively. The catalyst can be represented by the formula MEₓ, where M is a Group VI transition metal, E is a non-metal element, and x is at least 2, such as 2 or at least 3, 4, or 5. For example, M can be selected from Cr, Mo, and W and/or E can be selected from non-metal elements, such as B, C, N, S, Se, Te, and P. In some cases, the Group VI transition metal chalcogenide is selected the group of MoSₓ, MoSeₓ, WSeₓ, and WSₓ. The Group VI transition metal chalcogenide can be amorphous or crystalline.

The Group VI transition metal chalcogenide catalyst can be deposited, coated, or integrated on a base electrode. The base electrode can be an inert electrode composed of, for example, gold, platinum, glassy carbon, graphite, nanocarbon material, indium-tin oxide (ITO), or fluorine-doped tin oxide (FTO). The base electrode can be a transparent conducting electrode (TCE). The Group VI transition metal chalcogenide catalyst can be coated on the base electrode by any method that provides a redox potential of hydride formation that is more negative than the redox potential of the cofactor to be reduced (i.e., so that the catalyst can hydrogenate the cofactor). For example, the catalyst can be prepared by electrodeposition, sputter-coating, drop-coating, dip-coating or spin-coating.

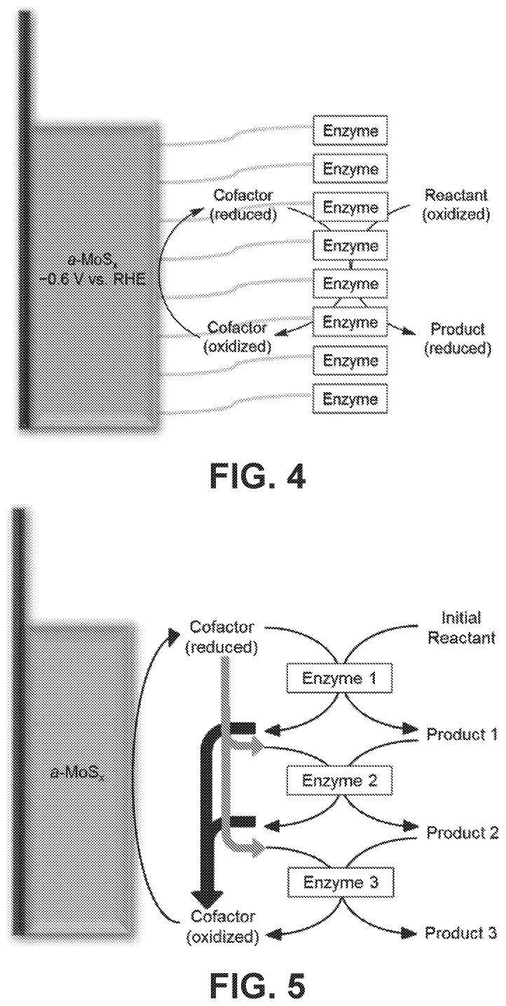

In some cases, an electrode of the present disclosure further comprises a conjugated enzyme. For example, the cofactor-dependent oxidoreductase can be conjugated to the surface of the electrode. In some cases, the surface of the catalyst is functionalized with at least one linker group. The linker group can be selected based on based on the non-metal element of the chalcogenide. A suitable linker group can be any bifunctional organic molecule that confers a functional moiety that can be used to immobilize a protein to the catalyst. For example, when the non-metal is sulfur, a suitable linker provide for direct conjugation to the sulfur through C—S bond formation. Functional groups on the other end of the linker can be then used to immobilize proteins. See for example, FIG. 4.

The cofactor-dependent oxidoreductase can be directly conjugated to the catalyst. For example, conjugation can be achieved via disulfide-bond formation between the thiol functionality of native or engineered cysteine on the enzyme and a thiol moiety present on the catalyst surface; covalent bond formation between the thiol functionality of an enzyme's native or engineered cysteine and a cysteine-reactive functionality present on the catalyst surface including but not limited to maleimide, haloacteamide, alkene (for radical initiator or photosensitizer promoted thiol-ene reaction) and alkyne (for radical initiator or photosensitizer promoted thiol-yne reaction) linkers; amide bond formation through native or non-native chemical ligation between an enzyme's native or engineered N-terminal cysteine and a thioester present on the catalyst surface; covalent bond formation between the nucleophilic amine functionality of an enzyme's lysine or N-terminus and an electrophilic functionality present on the catalyst surface including but not limited to activated acids (e.g., acyl halides, NHS esters, sulfo-NHS esters, O-acylisourea from carbodiimide-mediated activation of carboxylic acid moieties, and mixed anhydrides or acylimidazols from N,N′-carbonyldiimidazole or N,N′-disuccinimidyl carbonate mediated activation of carboxylic acid moieties), activated carbamates (e.g. from N,N′-carbonyldiimidazole or N,N′-disuccinimidyl carbonate mediated activation of amine moieties), activated carbonates (e.g., from N,N′-carbonyldiimidazole or N,N′-disuccinimidyl carbonate mediated activation of hydroxy moieties), vinyl sulfones, isocyanates, isothiocyanates, and squaric acids; covalent bond formation through imine formation or reductive amination reactions (e.g., in presence of sodium cyanoborohydride) between the nucleophilic amine functionality of an enzyme's lysine or N-terminus and the carbonyl moiety (e.g. of an aldehyde or ketone) present on the catalyst surface; amide bond formation between the carboxylate functionality of an enzyme's aspartate, glutamate or C-terminus activated by conversion with a carbodiimide (e.g. EDC) or phosgene derived (e.g. CDI or DSC) reagent and a nucleophilic functionality present on the catalyst surface including but not limited to amine and alcohol moieties; ester bond formation between the carboxylate functionality of an enzyme's aspartate, glutamate or C-terminus and a diazoalkane or diazoacetyl functionality present on the catalyst surface; bioorthogonal conjugation (e.g. carbonyl condensation, Staudinger ligation, strain-promoted [3+2] cycloaddition, dipolar cycloaddition reactions, inverse electron demand Diels-Alder cycloadditions, transition metal catalyzed cycloadditions, 1,3-photoclick cycloadditions, and transition metal catalyzed C—C coupling reactions) between a reactive group on the enzyme introduced by prior chemical conjugation or incorporation of an unnatural amino acid (UAA) and the complementary group present on the catalyst surface.

In one or more embodiments, the cofactor-dependent oxidoreductase is engineered for conjugation to the catalyst. For example, the enzyme can be synthesized by heterologous expression to include an engineered peptide sequence that facilitates enzymatic conjugation to the catalyst. In some cases the surface of the catalyst is modified with a complementary peptide sequence. For example, the catalyst surface can be modified by methods such as sortase-, subtiligase- and spyLigase-catalyzed transpeptidation; transglutaminase-catalyzed amide-bond formation; and lipoic acid ligase-catalyze acylation.

In one or more embodiments, the cofactor-dependent oxidoreductase is conjugated to the surface of the catalyst via a biotin/streptavidin-type interaction (e.g., chemical conjugation of biotin with the protein and its binding to a tetrameric streptavidin or streptavidin-like protein; incorporation of a non-canonic amino acid with a biotin side chain into the protein and its and binding to a tetrameric streptavidin or streptavidin-like protein; BirA-catalyzed enzymatic conjugation of biotin with the AviTag™ of an correspondingly engineered protein and its binding to a tetrameric streptavidin or streptavidin-like protein; and genetic fusion of a strep-tag type sequence with an protein and its binding to a tetrameric streptavidin or streptavidin-like protein).

The aqueous electrolyte solution can be configured to stabilize the cofactor-dependent oxidoreductase and permit electrocatalytic cofactor reduction. The electrolyte is primarily composed of water with a conductive ionic species, such as but not limited to potassium phosphate, sodium phosphate, potassium perchlorate, or any other ionic species that can provide conductivity to an aqueous solution. The electrolyte is also conducive to enzyme survival so that enzyme denaturation is substantially inhibited. For example, the concentration of organic solvent is controlled to avoid denaturation. In some cases, the aqueous electrolyte solution has an alkaline or neutral pH. For example, the pH can be maintained between about 6 and about 9. In some cases, the aqueous electrolyte solution contains the cofactor-dependent oxidoreductase. The cofactor-dependent oxidoreductase can be solubilized in the electrolyte solution.

A Group VI transition metal chalcogenide catalyst of the present disclosure can be used to improve the rate of an oxidoreductase-catalyzed reaction. The rate of the reaction is improved compared to the rate of a corresponding oxidoreductase-catalyzed reaction performed without regenerating the oxidized cofactor. A “corresponding” reaction is one that is carried out under otherwise identical conditions. The method can include reacting an oxidoreductase and a substrate thereof in the presence of an oxidoreductase cofactor, whereby the substrate is converted to a first product and the cofactor is oxidized and regenerating the oxidized cofactor according to the method described above.

The oxidoreductase can be selected from the group consisting of nicotinamide-dependent oxidoreductases, NADH-dependent oxidoreductases, NADPH-dependent oxidoreductases, and FADH₂-dependent oxidoreductases. For example, the oxidoreductase can be selected from alcohol dehydrogenases, aldehyde dehydrogenases, ene reductases, amino acid dehydrogenases, oxidoreductases of CH—NH groups, nitrate reductases, oxidoreductases acting on a sulfur group, dehydrogenases of diphenols, peroxidases, hydrogenases, oxygenases, monooxygenases, oxidoreductases of metal ions, oxidoreductases acting on CH or CH₂ groups, oxidoreductases of iron-sulfur proteins and of flavodoxin, reductive dehalogenases, and oxidoreductases reducing a C—O—C group.

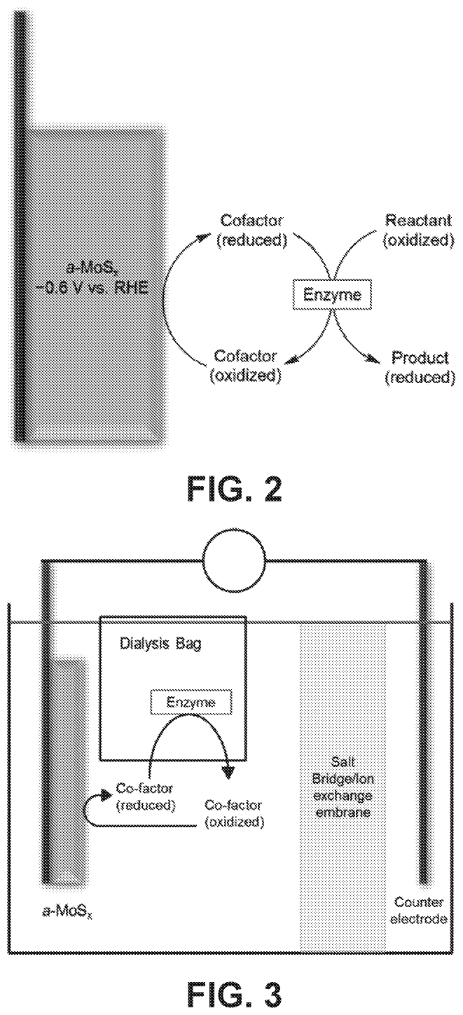

A system for practicing the methods of the present disclosure can include a reaction vessel that is configured to contain an electrode comprising a Group VI transition metal chalcogenide catalyst and an aqueous electrolyte solution. FIG. 2 illustrates an embodiment of a system and method of using a Group VI transition metal chalcogenide catalyst of electrochemically regeneration of an enzyme cofactor. The system includes a solubilized enzyme (i.e., the cofactor-dependent oxidoreductase is soluble in the aqueous electrolyte solution). The cofactor is reduced on the electrode deposited with the Group VI transition metal chalcogenide catalyst, here, amorphous molybdenum sulfide (a-MoSₓ), by holding at a potential cathodic enough to form a metal hydride and more negative than the redox potential of the cofactor. The reduced cofactor can then be utilized by the soluble enzyme to convert a substrate (also referred to as “reactant”) to a desired product. For example, the reduced cofactor can be used by solubilized alcohol dehydrogenase to hydrogenate benzaldehyde to benzyl alcohol.

Turning to FIG. 3, the electrode with the Group VI transition metal chalcogenide catalyst is placed into an cell with electrolyte containing oxidized (or non-reduced) cofactor (NAD⁺, NADP⁺, FAD), the cofactor-dependent oxidoreductase and a substrate thereof, and a reducing potential is applied. The electrode is connected to a counter electrode, optionally separated from the rest of the electrolyte in a separated compartment, which is connected to the rest of the cell by salt bridge or anionic exchange membrane. This separator can be impermeable to cofactor, enzyme, and substrate diffusion. The liquid present in the counter electrode compartment is any liquid solution that allows conduction. In an embodiment, the liquid has the same composition as the aqueous electrolyte solution. The amount of reducing current or potential applied is such that the electrode reduces the cofactor (i.e., a reducing current or potential where hydride formation occurs). The cofactor reduction drives the reduction reaction itself. The product can be isolated via any separation technique suitable for use with enzyme catalyzed reactions.

As shown in FIG. 3, the reduced cofactor diffuses through a dialysis bag to the enzyme and the reaction proceeds. The oxidized cofactor then diffuses back through the membrane to the catalytic electrode (here, amorphous molybdenum sulfide (a-MoSx)). The system shown includes a counter electrode and a salt bridge/ion exchange membrane for electrical neutrality. In some cases, the enzyme is immobilized on a membrane (e.g., a hollow fiber membrane). The membrane is permeable to cofactor diffusion.

FIG. 4 illustrates another embodiment of a system and method of using a Group VI transition metal chalcogenide catalyst (as shown, a-MoSx). In this case, a plurality of enzymes molecules are conjugated to the surface of the electrode, forming a biocatalytic electrode. The cofactor is soluble and is reduced on the surface of the electrode, to become available for the conjugated oxidoreductase. The enzyme can be conjugated to the electrode by any method described above.

FIG. 5 shows a further embodiment of the present disclosure. In this case, a first enzyme converts an initial substrate into a first product, which is a substrate for a second enzyme to convert into a second product; and the second product is a substrate for a third enzyme. Thus, the system is configured for multi-step biocatalytic reactions. One or more of the first, second, and third enzymes utilize a reduced cofactor, thereby oxidizing it. The cofactor can be reduced by a Group VI transition metal chalcogenide electrocatalyst (MoSx), here amorphous molybdenum sulfide (a-MoSx). In the cascade shown, all three enzymes require reduced cofactor in order to catalyze their respective reactions. In some cases, multiple different enzymes are part of a common catalytic pathway or an “enzyme cascade” (i.e., a series of enzymes in which the product of one enzyme is the substrate for the next). One or more of the enzymes can be immobilized on the surface of the electrode as described above, or soluble in the aqueous electrolyte solution. The soluble enzymes can be separated from the electrode by a membrane as described above. The cascade can be initiated by introduction of the first substrate to the aqueous electrolyte solution.

### EXAMPLES

The following Examples are intended to illustrate the above invention and should not be construed as to narrow its scope. Numerous variations and modifications may be made while remaining within the scope of the invention.

These examples provide the first direct experimental evidence of an immediate role for Mo in heterogeneous H₂ evolution, placing a paramagnetic Mo center, as opposed to its partner atoms, as an HER-active site with uniquely high activity for hydride formation and transfer. This mechanistic finding also reveals that Mo sulfides have potential as economic electrocatalysts for NADH regeneration in biocatalysis.

### Example 1: Mo³⁺ Hydride as the Common Origin of Efficient H₂ Evolution and Selective NADH Regeneration Activity in Molybdenum Sulfide Electrocatalysts

### Introduction

Hydride transfers are key to a number of economically and environmentally important reactions, including H₂ evolution and NADH regeneration. Therefore, the electrochemical generation of reactive hydrides has the potential to drive the electrification of chemical reactions to improve their sustainability for a green economy. Catalysts containing molybdenum (Mo) have recently been recognized as amongst the most promising non-precious catalysts for H₂ evolution, but the mechanism of Mo in conferring this activity remains debated. A modified EPR setup was used to demonstrate the presence and catalytic role of a trapped Mo³⁺ hydride in amorphous Mo sulfide (a-MoSₓ), one of the most active non-noble H₂ evolution catalysts yet reported. The results show that this hydride is active for the selective electrochemical hydrogenation of the biologically important energy carrier NAD to its active NADH form and can, therefore, be utilized for biocatalysis. Furthermore, the data supports applying other HER-active forms of Mo sulfide for biocatalysis.

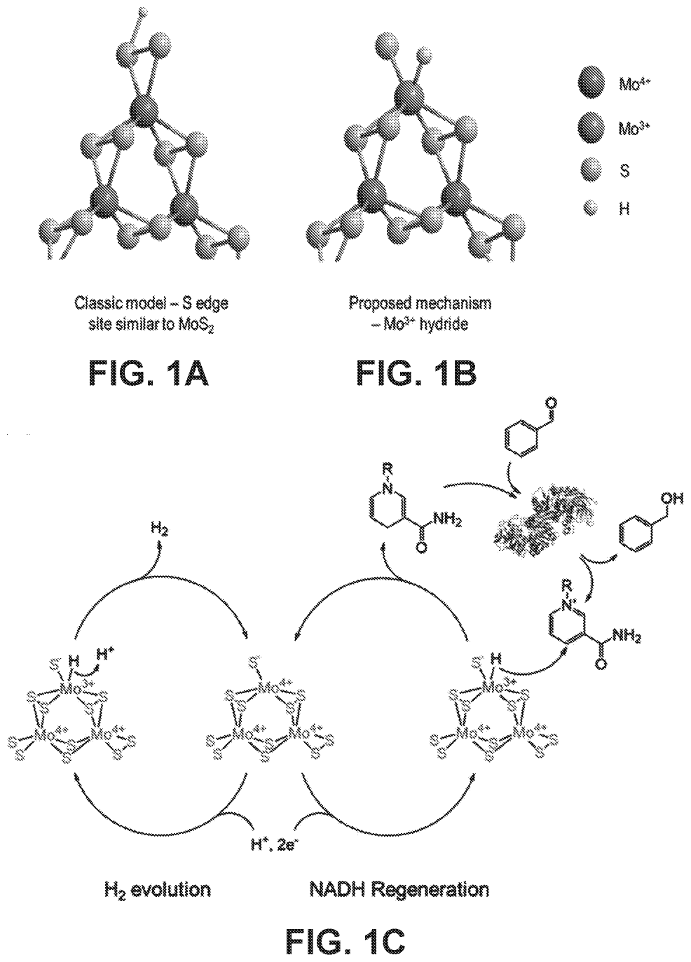

Despite the prevalence of Mo in heterogeneous HER electrocatalysts, a direct role for Mo in the reaction (such as the formation of a metal hydride) remained experimentally unresolved. In Mo sulfides, a thiol-like mechanism is instead favored due to the strong hydrogen binding energy of metallic Mo which should lead to poor HER activity (FIG. 1A). However, the existence of HER-active single-atom molecular Mo catalysts that are non-zero valent and sulfide-free demonstrates that chemically reactive H₂ intermediates can be formed on Mo active sites. The involvement of a metal hydride intermediate in Mo-based catalysts would provide an explanation for the uniquely high activity for HER in these systems as well as assist in the design of future catalysts for the HER and electrochemical hydride transfers.

This work describes the specific reduction of NAD and its analogue N-methyl nicotinamide (NMN), to their high-energy dihydropyridine derivatives using Mo sulfide catalysts, demonstrating the hydride nature of the Mo—H bond and its ability to catalyze transfer hydrogenation reactions using water as a hydride source.

### Results

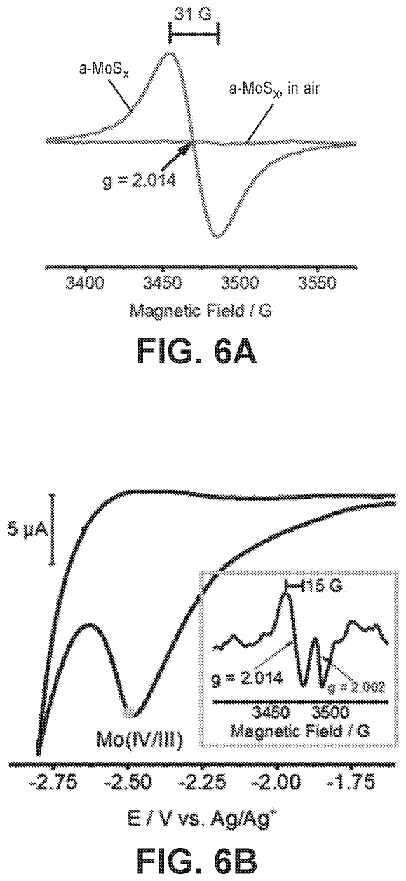

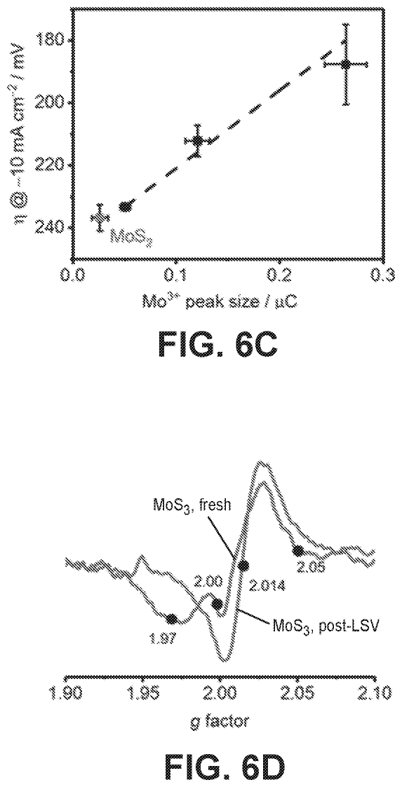

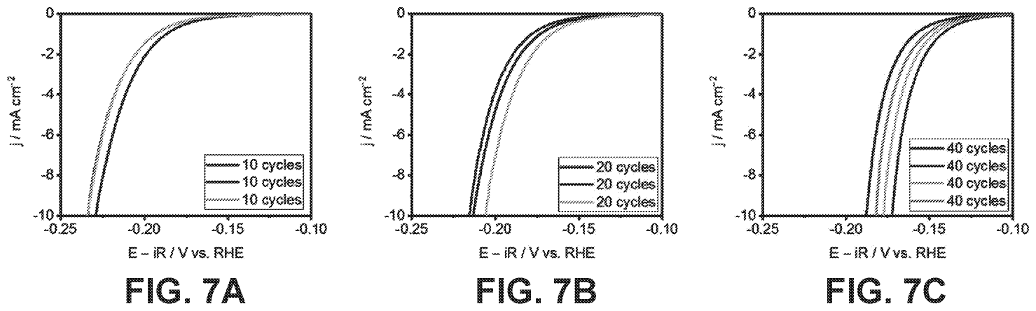

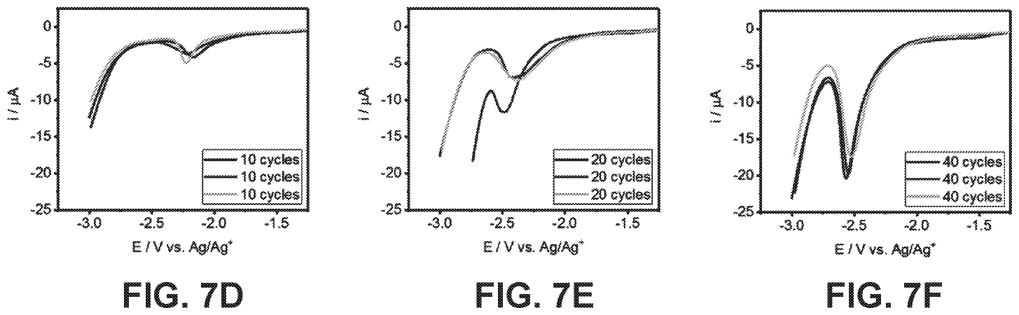

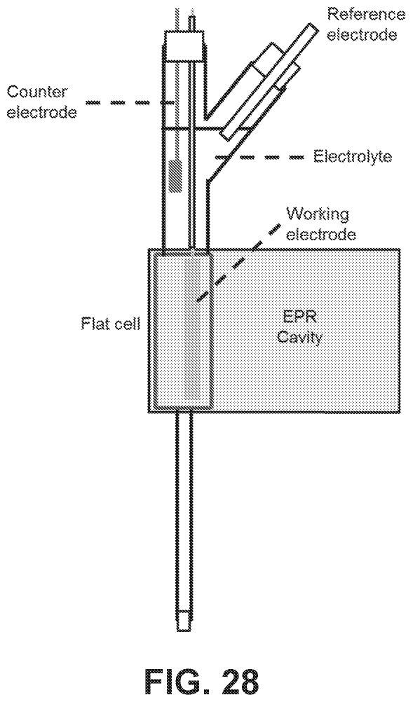

The Mo³⁺ hydride species was captured during the electrodeposition of a-MoSₓ from MoS₄²⁻ solutions during cyclic voltammetry (CV), in which MoS₃ is deposited at anodic potentials and subsequently reduced to a-MoSₓ at cathodic potentials close to the onset of the HER (FIG. 7). A EPR setup was modified to measure the oxidation states of paramagnetic species in complex electrocatalysts by combining a standard Wilmad-LabGlass electrolytic EPR flat cell with flat wire electrodes. Catalyst was deposited on the flat wire so that it fits into the flat cell, resulting in minimal microwave interactions with both electrolyte and metal. Consequently, signal from catalyst deposited on the wire can be acquired (FIG. 28). To prepare a sample suitable for EPR measurements, a-MoSₓ was deposited on the flat Au electrode and loaded into the EPR flat cell filled with tetrahydrofuran (THF). If the deposition was ended on the cathodic edge of the scan range, the resulting catalyst exhibited an isotropic EPR spectrum as shown in FIG. 6A. The spectrum itself was centered at 2.014, typical of the characteristic g-value for Mo³⁺, and had an average peak separation of 31 G, making it dissimilar to sulfide-related paramagnetic states in MoS₂ that have smaller peak separations (≤10 G) and g-values at 2.002. The EPR signal of the electrodes rapidly disappeared if the electrode was not immediately isolated under dry, O₂-free conditions, reflecting the metastability of Mo³⁺.

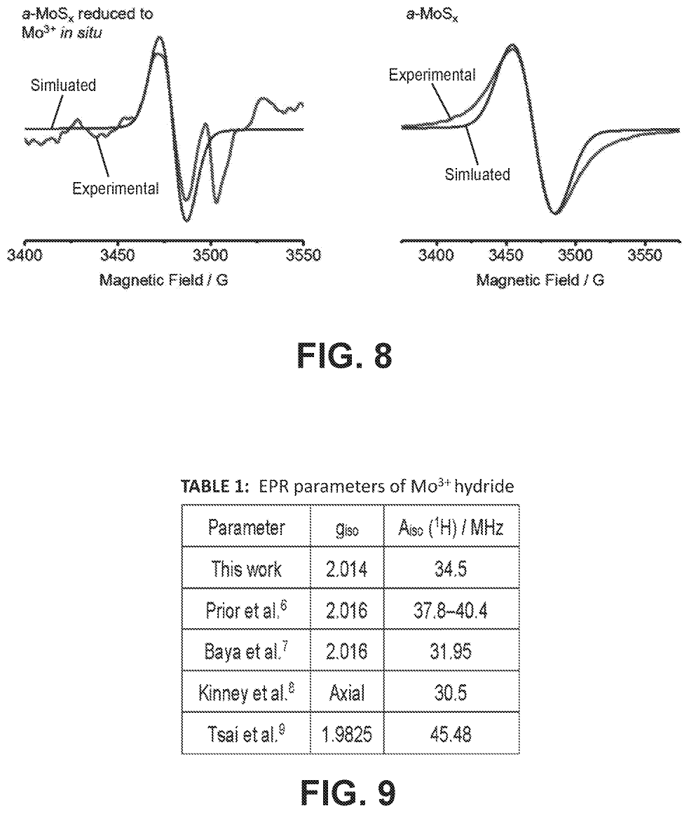

Although the Mo³⁺ EPR signal could be lost upon oxidation, it could also be partly restored if a-MoSₓ was reduced in organic electrolyte (0.2 M Bu₄N PF₆/THF, FIG. 6B) within the cavity of the EPR spectrometer, revealing the re-emergence of the Mo³⁺ signal with a width of 15 G (inset). The contrast in peak-to-peak width between the original signal and the reduced form in aprotic electrolyte indicates the presence of hyperfine coupling between a spin-active nuclei and the Mo³⁺ center. As less than 1% of naturally occurring S has a nuclear spin, the only other possible candidate atom was H, revealing the presence of a hydride directly bound to Mo³⁺. Further confirmation of the bound hydride was modeled using EasySpin, where a hydride hyperfine coupling constant of −34.5 was fit to the peak, within range of previously reported Mo³⁺ hydrides (FIGS. 8 and 9 (Table 1)). These EPR spectra therefore represent the first direct evidence for the formation of a hydride associated with Mo³⁺ in a-MoSₓ.

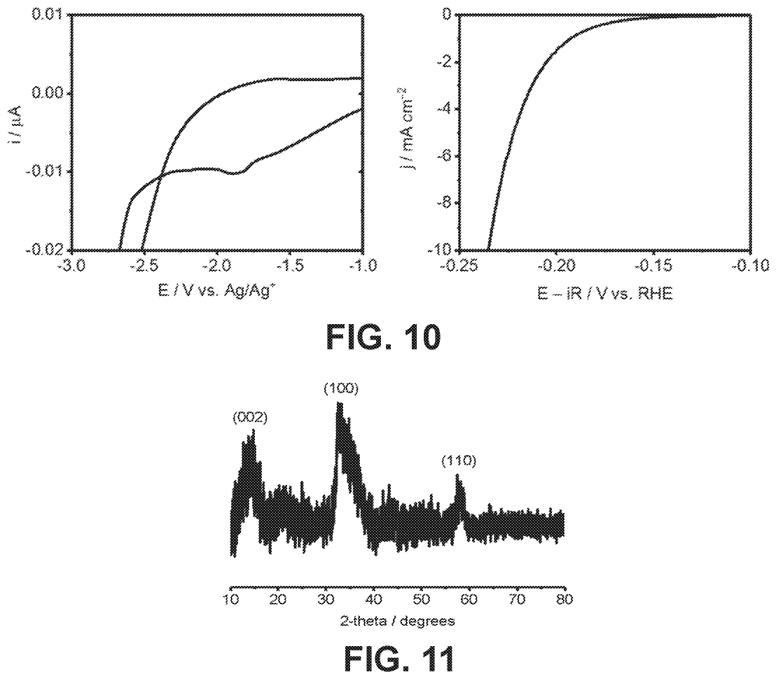

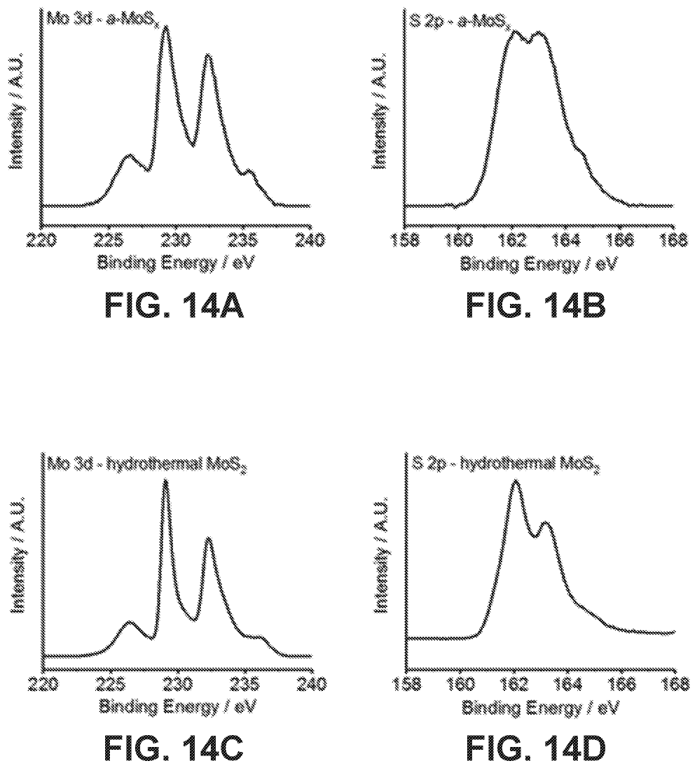

Only one major reduction peak was observed during a cathodic sweep from open circuit potential, demonstrating that the resting state of the catalyst was Mo⁴⁺, in line with X-ray photoelectron studies. Coulometric measurements of the reduction event revealed that it corresponded to 40% of the Mo present in the a-MoSₓ catalyst as determined by inductively coupled plasma-optical emission spectroscopy (ICP-OES), suggesting that 40% of Mo in a-MoSₓ can be converted to the Mo³⁺ state. In comparison, the Mo hydride species in freshly deposited a-MoSₓ represented 2% of the total Mo as determined by spin counting. Furthermore, by using a-MoSₓ electrodes with increasing deposition amounts, the area of the peak as determined using coulometry was found to form a linear correlation with the activity of the a-MoSₓ itself as measured by overpotential at −10 mA cm⁻² in 0.05 M H₂SO₄ (FIG. 6c). This relationship between Mo³⁺ peak size and HER activity was also found to be relevant to MoS₂, as HER-active MoS₂ prepared by autoclave synthesis (for MoS₂) was found to fit the trend (FIGS. 10-11) (The pattern matches well to reported patterns for MoS₂ nanosheets). Alternately, epitaxially grown, single-crystal (less defective) MoS₂ had no Mo³⁺ peak nor significant HER activity (FIGS. 14A-D). Therefore, the amount of reducible Mo³⁺ correlates directly to the HER activity of a given Mo sulfide electrode and can be used as a quantifiable benchmark for the HER activity of Mo sulfides.

The existence of an isotropic Mo³⁺ hydride in a-MoSₓ provides valuable insights into the structure of the catalyst. For example, Mo⁵⁺ in anodically deposited Mo₃S₁₃ is coordinated by a number of theoretically equivalent S atoms, but does not have an isotropic EPR signal (FIG. 6D), reflecting the inequivalent positioning of S atoms around the Mo center. In contrast, the isotropic Mo³⁺ spectrum correlates with the equivalent coordination of atoms (H and S) around the Mo center. Since a-MoSₓ (at least initially) resembles Mo₃S₁₃ clusters, the only possibility that could explain the isotropic hydride signal is the presence of a hydride within a Mo₃S₁₁(H) cluster. The loss of two S atoms—the apical S as well as one of the disulfide S—results in Mo being equivalently coordinated by six atoms (five S and one H) while accounting for the presence of a metal hydride. The transformation between Mo₃S₁₃ and Mo₃S₁₁(H) is labile, as the Mo⁵⁺ EPR signal in anodically-deposited MoS₃ could be rapidly converted to the Mo³⁺ hydride signal upon a single linear potential sweep to the HER potential in aqueous electrolyte.

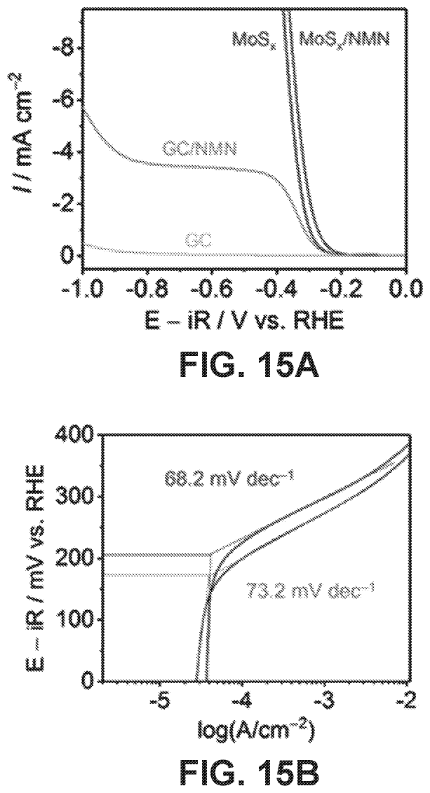

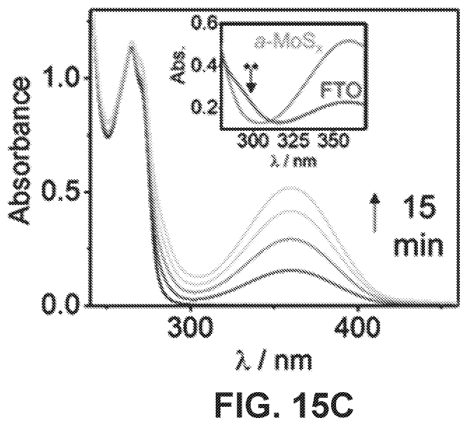

The role for a hydride intermediate during the HER on a-MoSₓ is not just of fundamental interest; Mo hydrides have also been demonstrated as amongst effective non-noble metal hydrogenation catalysts, suggesting that a-MoSₓ could be used in electrocatalytic transfer hydrogenation reactions using water as a hydride source, particularly for the biocatalytically-significant recycling of NADH. The reduction of N-methyl nicotinamide (NMN), an analogue of NAD, which is also hydrogenated to a 1,4-dihydropyridine, was then studied. However, NMN is more ideal for distinguishing reduction mechanisms as its single-electron transfer products have distinct spectroscopic (NMR and UV-vis) properties, unlike NAD. Electrochemical reduction of NMN in electrolyte (0.5 M K₂CO₃, pH 10, 5 mM NMN) by a-MoSₓ results in a 30 mV positive shift of the onset of catalysis compared to the HER (FIGS. 15A-C), which is reasonable given that NAD⁺ reduction to NADH has a less cathodic electrode potential compared to the HER. NMN reduction otherwise does not affect the activity of the HER, as evidenced by the similarity in linear sweep voltammograms between a-MoSₓ in NMN-containing and NMN-free electrolytes. In contrast, common electrode materials such as glassy carbon (GC) reduce NMN in two sequential reduction processes corresponding to the two different reduction mechanisms discussed above. Furthermore, the onset of the one-electron reduction happens close to the HER onset for a-MoSₓ. Therefore, the mechanism of NMN reduction (1,4-dihydropyridine or dimer followed by breakdown) can be used to determine the role and presence of a metal hydride.

To distinguish the two reduction mechanisms for the reaction on a-MoSₓ, UV-visible spectroscopy was used to analyze the NMN electrolyte before and after electrolysis using a-MoSₓ. The 1,4-dihydropyridine absorbs strongly at 360 nm while the dimer and its decomposition products have absorption peaks centered at both 360 and 298 nm. The NMN electrolyte gradually formed a peak at 360 nm when electrolyzed with a-MoSₓ at a current density of −10 mA cm⁻² (η=˜−450 mV) with no prominent features at 298 nm being observed (FIG. 6C). In comparison, a fluorine-doped tin oxide (FTO) electrode electrolyzed at the same potential produced both a less intense 360 nm peak as well as the 298 nm dimer decomposition product peak (inset). The 360 nm peak was also less intense with electrolyte from FTO as compared to a-MoSₓ.

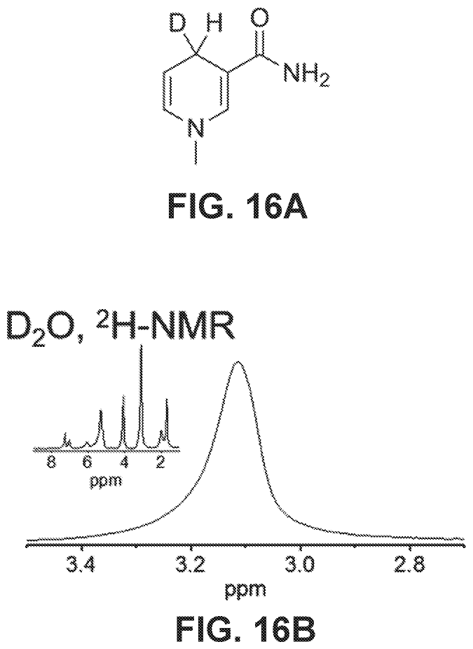

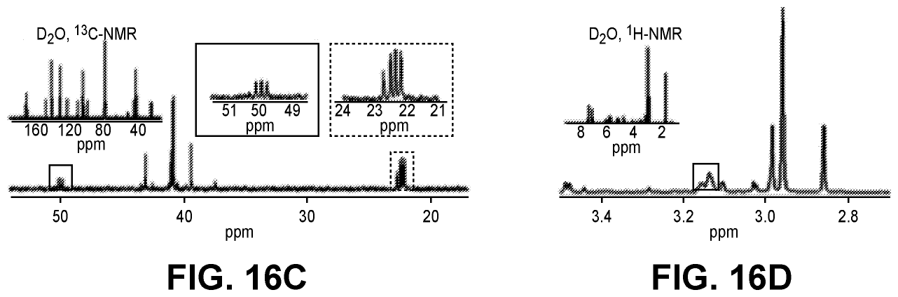

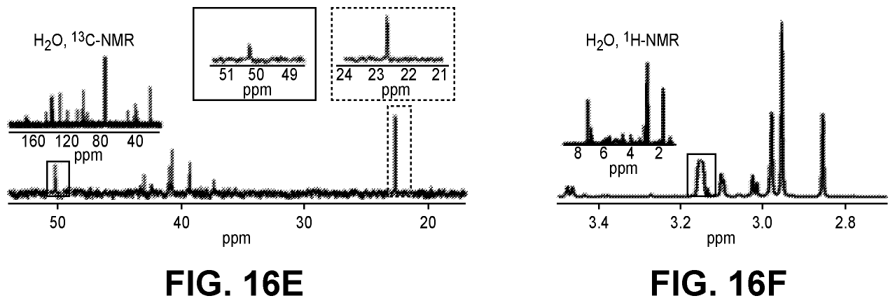

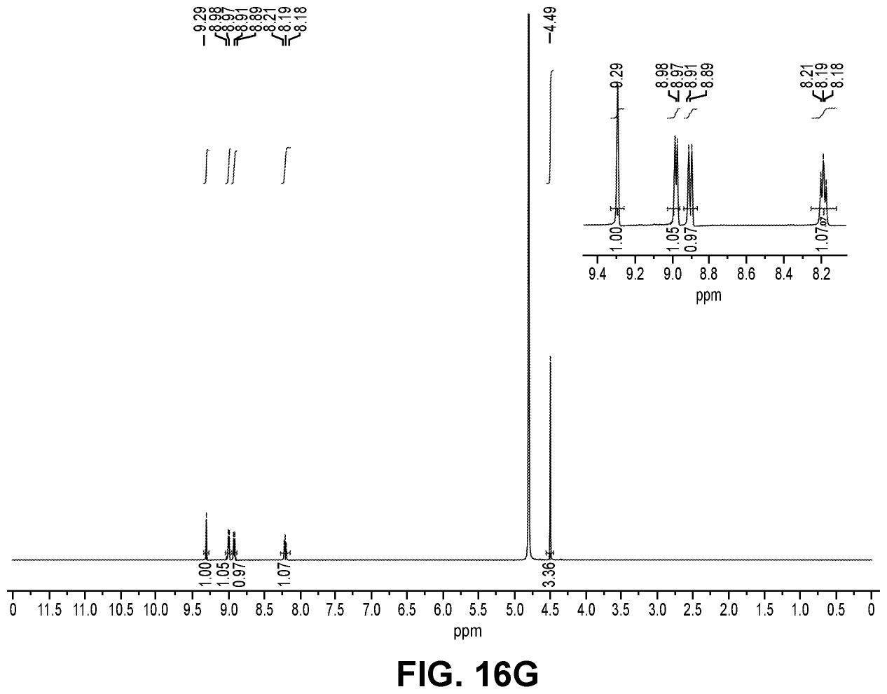

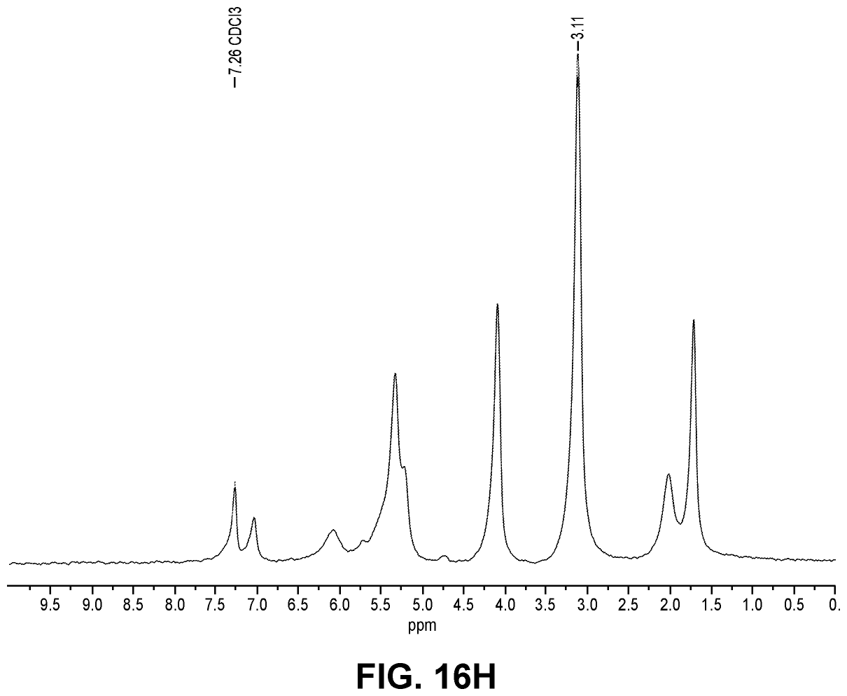

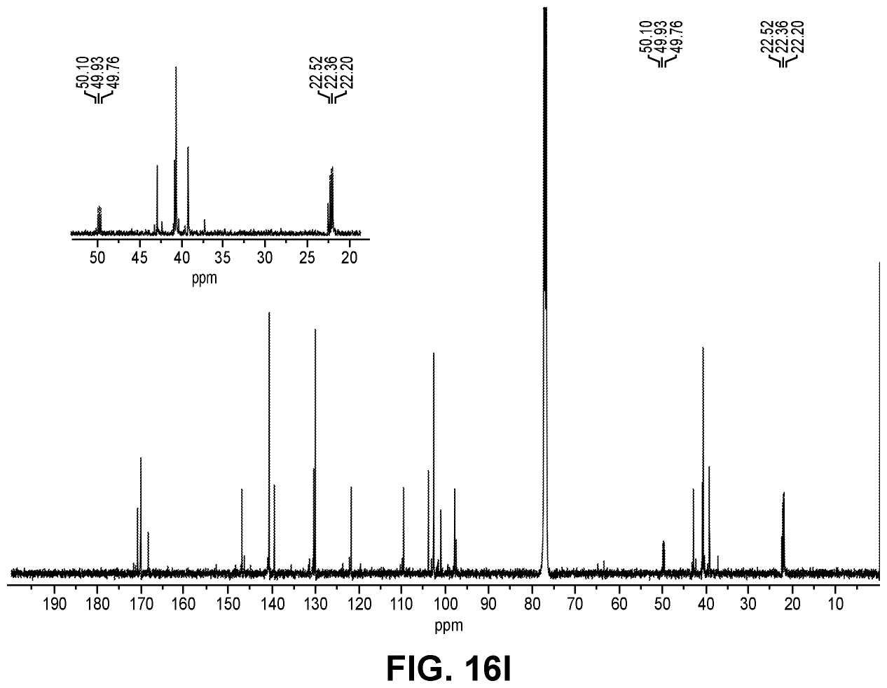

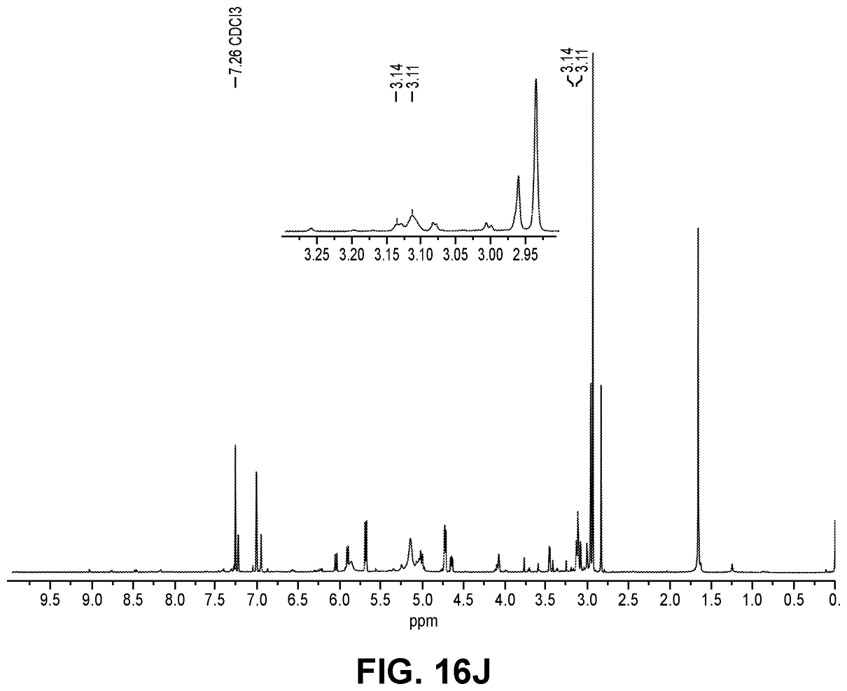

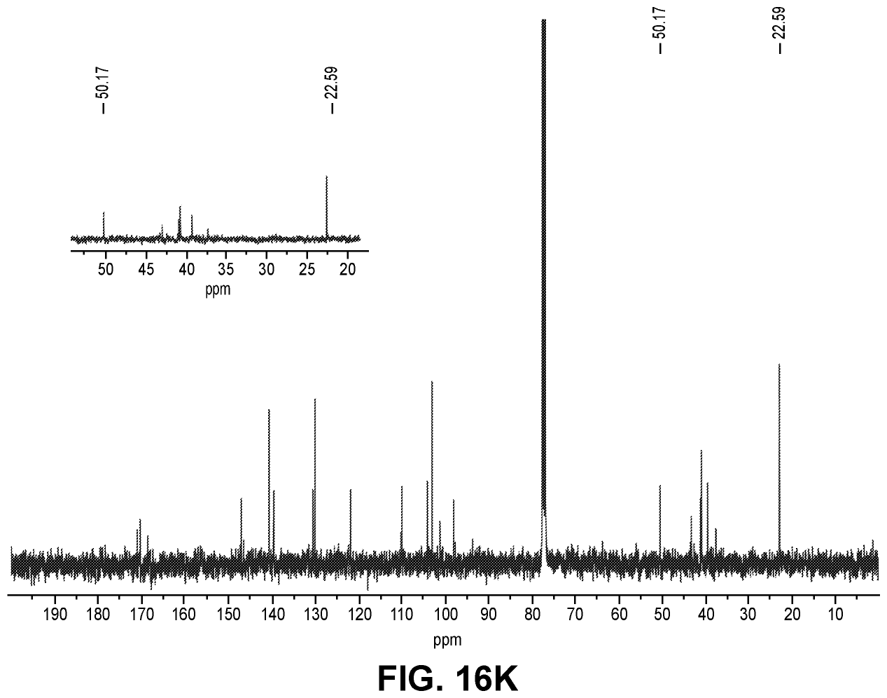

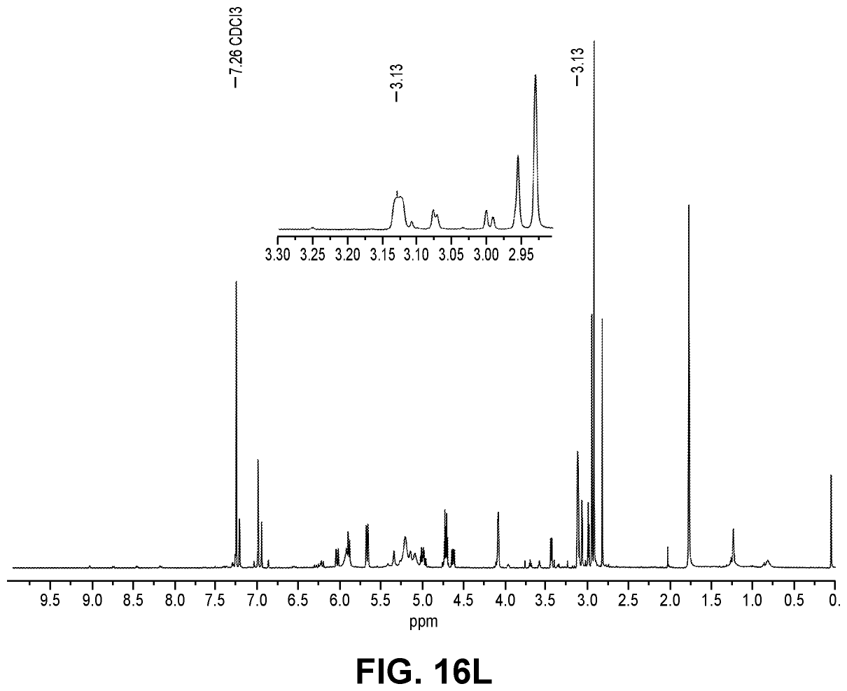

As a final confirmation that a-MoSₓ directly reduces NMN to the 1,4-dihydropyridine derivative without proceeding through single-electron transfer, the electrochemical reduction was additionally carried out in both the normal electrolyte as well as deuterated solution (D₂O/K₂CO₃/adjusted to pH 10 using D₂SO₄) followed by nuclear magnetic resonance (NMR) spectroscopy of the isolated product. Deuteration at C4 of NMN should lead to the formation of a CDH motif (FIG. 16A), resulting in the appearance of a triplet peak at the given NMR shift. Indeed, a triplet peak formed at 22.36 ppm in the ¹³C NMR spectra that was replaced by a singlet peak in the otherwise identical spectra of the product of the H₂O reaction (FIGS. 16C and E). A less intense triplet was also observed at 49.93 ppm, suggesting that the CDH motif was formed at other carbons as well. Deuterated species were found in the ²H-NMR spectrum of the isolated product; however, the 3.11 ppm peak corresponding to a deuterated C4 in the dihydropyridine structure was the most intense, confirming that the major hydrogenation product was the 1,4-dihydropyridine (FIG. 16B). The deuterated species were also observed in peak broadening between the ¹H-NMR spectra of the deuterated and non-deuterated products (FIGS. 16D and F). The direct formation of deuterated product without the dimerization side-reaction is confirmation that the Mo hydride is chemically reactive. (Full spectra at FIGS. 16G-L) These results demonstrate that Mo hydride, and specifically Mo³⁺ hydride, is the intermediate to H₂ evolution and nicotinamide reduction in Mo sulfides (FIG. 1C). Upon The evolution of H₂ evolution, Mo³⁺ is oxidized to Mo⁴⁺ and ultimately reduced to Mo³⁺ upon reformation of the hydride.

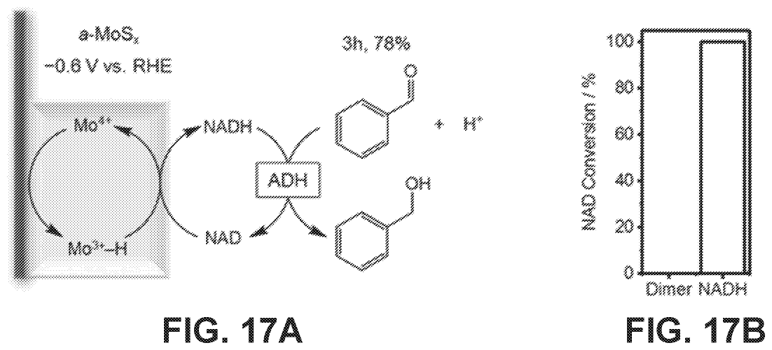

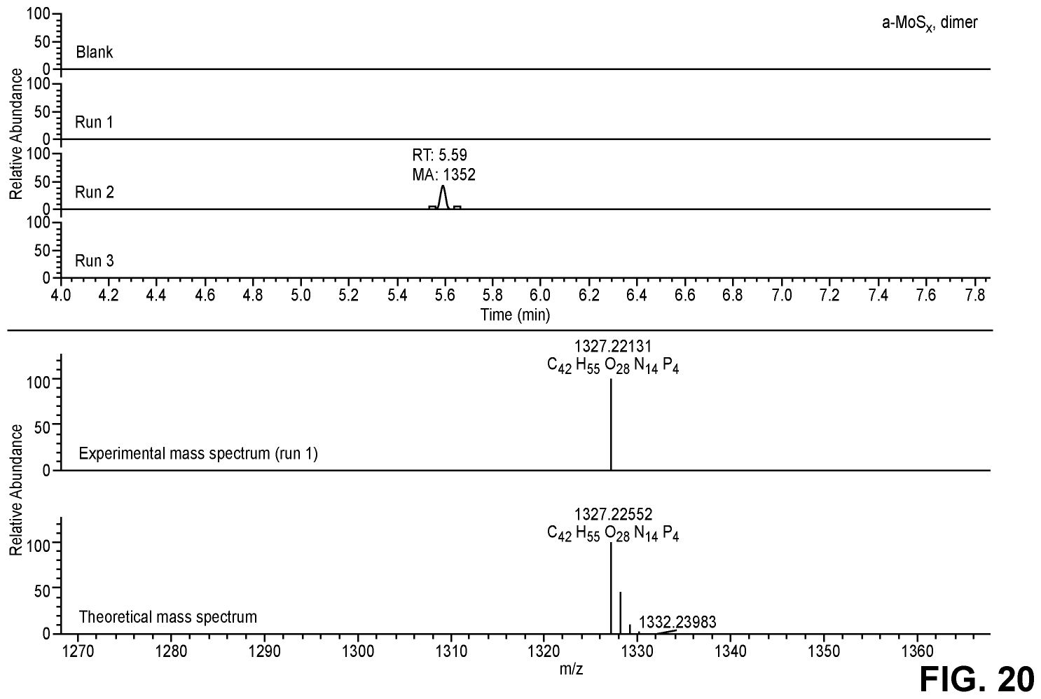

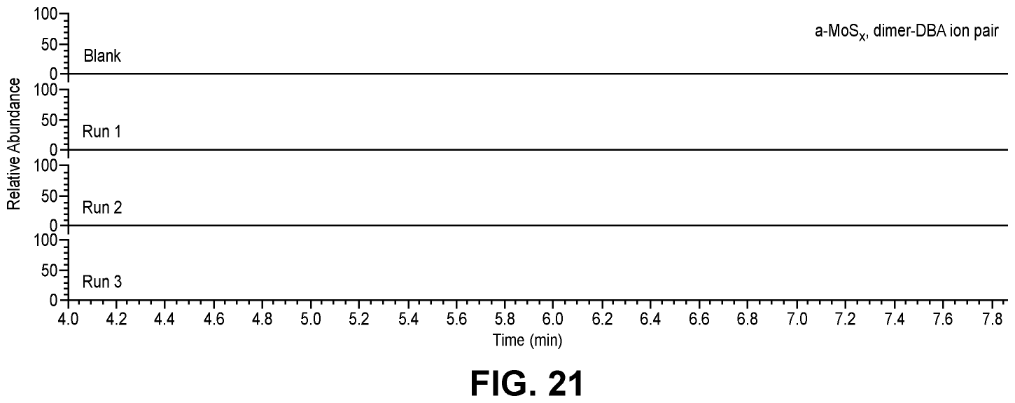

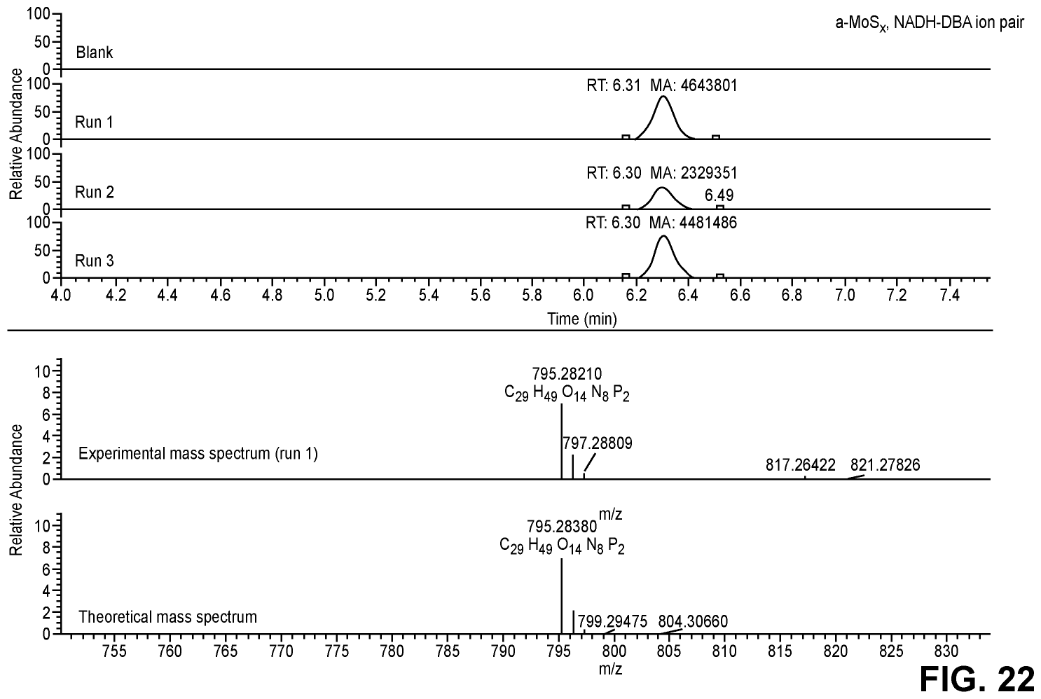

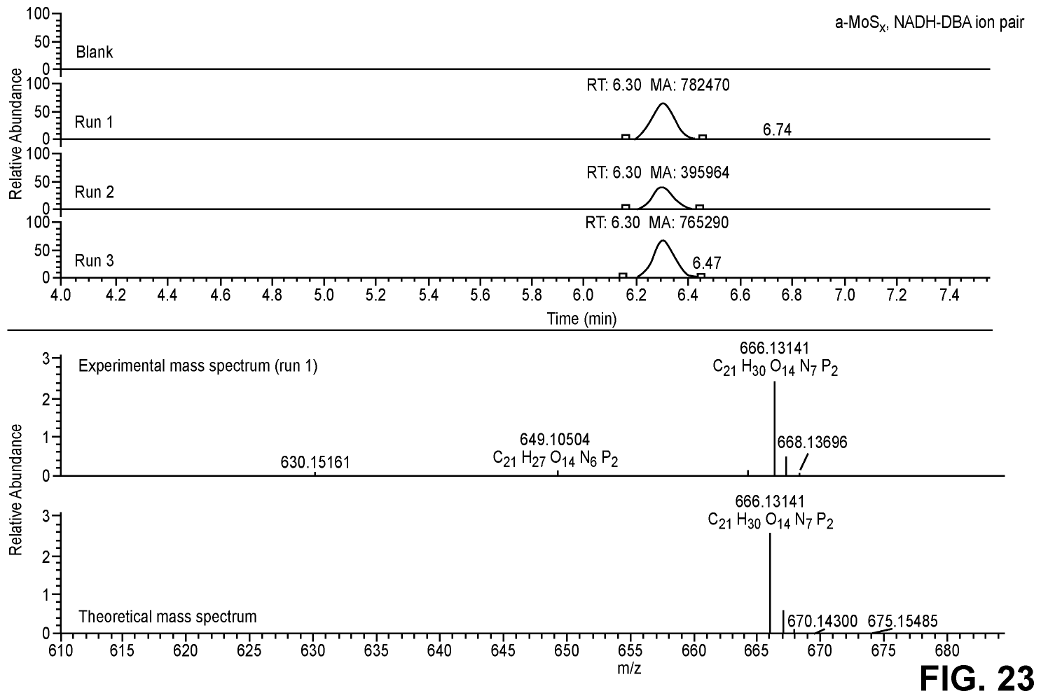

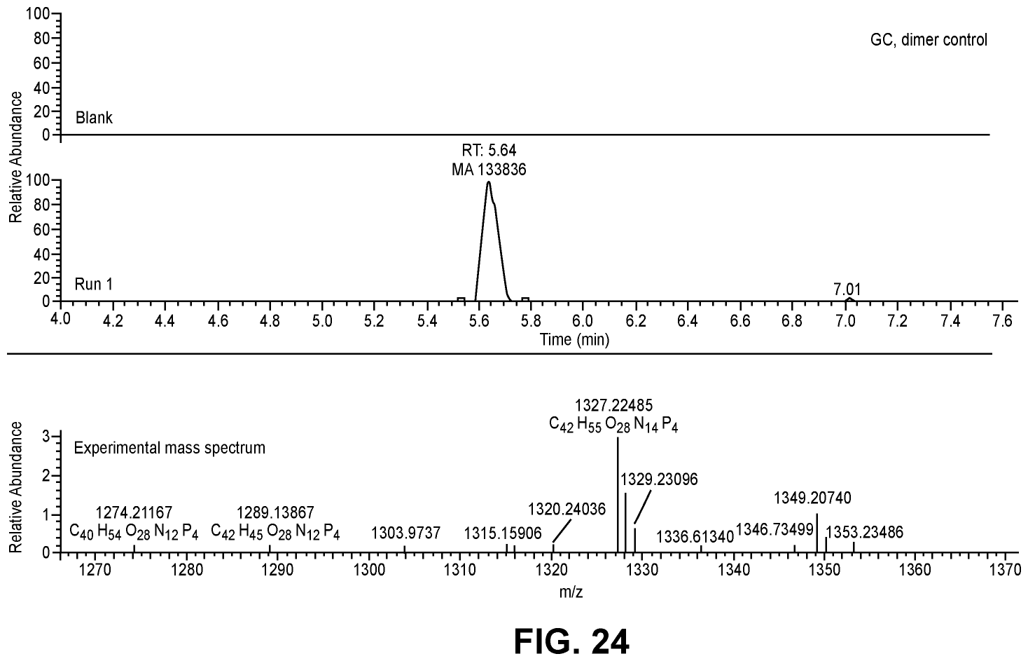

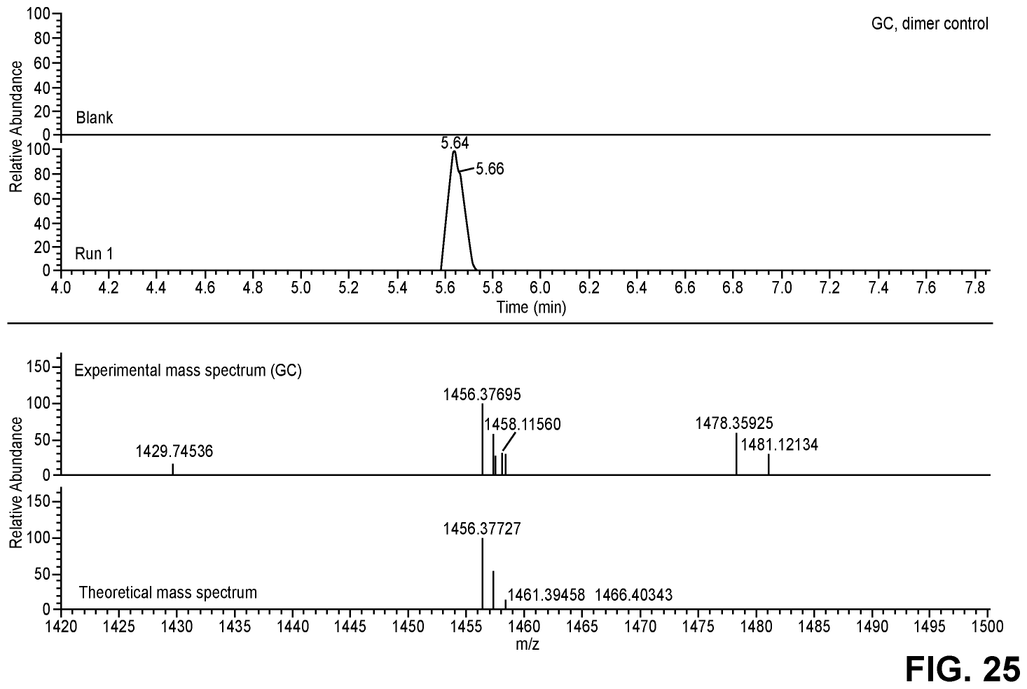

Based on the ability of a-MoSₓ to specifically hydrogenate NMN to its dihydropyridine derivative, the potential of a-MoSₓ for NADH regeneration was tested for the enzymatic hydrogenation of benzaldehyde to benzyl alcohol using alcohol dehydrogenase (ADH) from Saccharomyces cerevisiae (FIG. 17A). Selective regeneration of NADH without formation of the inactive (NAD)₂ dimer or inactive 1,2- and 1,6-dihydropyridine isoforms remains an important challenge in the application of electrocatalysis to biocatalysis, often necessitating the use of homogeneous, often noble metal-based mediators. The purity of NADH regenerated by a-MoSₓ was examined using an ultrahigh performance liquid chromatography system coupled to an orbitrap tribrid mass spectrometer (UHPLC-MS) in pH 9 electrolyte. After regeneration for 30 to 60 minutes at constant potential (−600 mV vs. RHE, average current ˜7 mA), the quantities of produced dimer were consistently marginal or undetectable (>0.1%, FIG. 17B and FIGS. 20-25), consistently reflecting the results of electrochemical NMN reduction where no dimer products were observed to form.

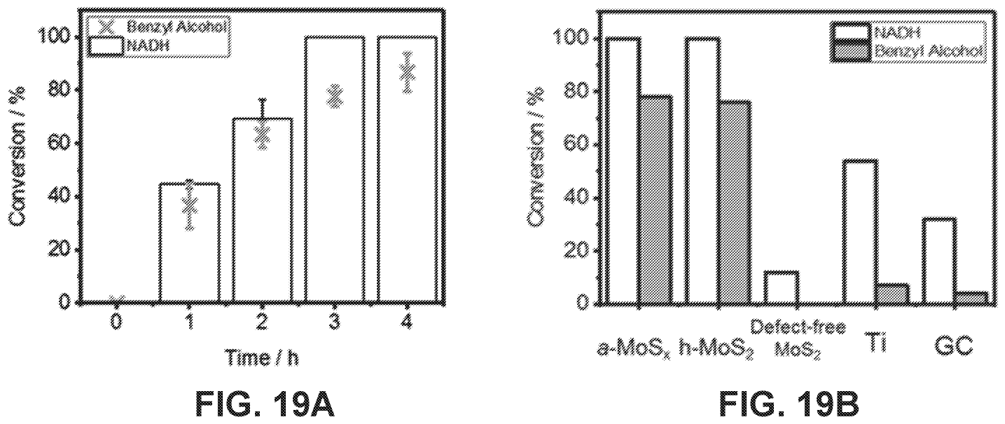

Since direct NADH regeneration proved promising, the utility of a-MoSₓ for direct biocatalysis was tested using the synthesis of benzyl alcohol from benzaldehyde in the presence of enzyme and tenfold excess of benzaldehyde (10 mM) to NAD (1 mM). After 3 hours, 78% conversion was achieved, rising to 87% over 4 hours (FIG. 19A). Therefore, over eight complete turnovers of NAD/NADH by a-MoSₓ were carried out over the course of the reaction. Simultaneously, NAD conversion to NADH increased as the reaction proceeded, reaching quantitative reduction at 3 hours as determined by two enzymatic NADH quantitation assays (Promega, Sigma-Aldrich), even as the rate of benzaldehyde conversion slowed. Naturally, NADH is rapidly consumed during the early stages of the reaction, but the enzymatic conversion of benzaldehyde appears to be the limiting step at later stages. Without being bound by theory, the appearance of foam formation during reaction suggested by co-generated H₂ was responsible for denaturing the enzyme over long time courses. a-MoSₓ also had no activity for benzaldehyde hydrogenation in the absence of enzyme or NAD, underlining its selectivity for the nicotinamide system. This specificity is important as it would allow for a-MoSₓ to be used in any future biocatalytic system without concerns that the catalyst could reduce other functional groups that might be present, obviating the need for protecting groups. The high yield of recycled NADH as determined by enzymatic assay clearly demonstrates that the alternate hydrogenation observed in the NMR experiments is avoided by the presence of the dinucleotide substituent.

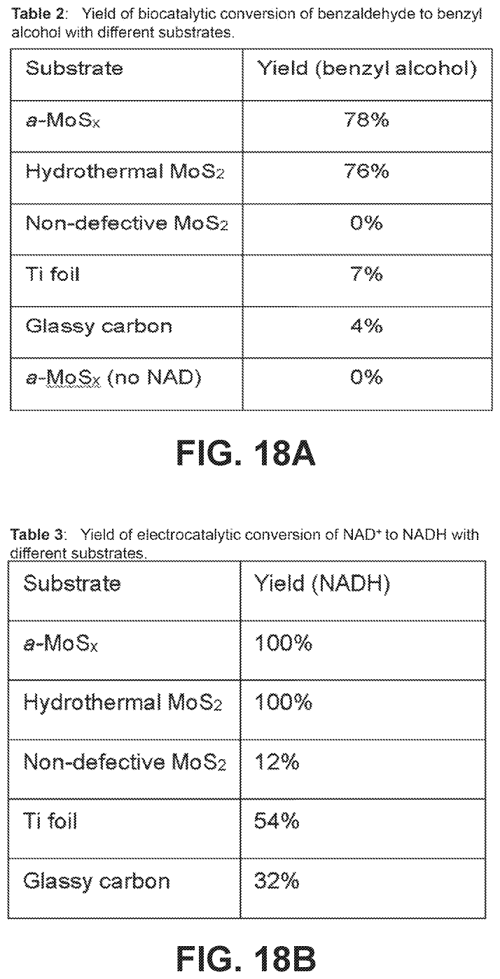

To confirm that the HER and NAD reduction arise from a common origin and mechanism, the ability of other Mo sulfides to carry out NAD reduction was tested in addition to previous experiments for the HER. Hydrothermally-prepared MoS₂ demonstrated similar activity (76% after 3 hours), but the epitaxially grown and continuously single-crystal (less defective) MoS₂ was inactive (FIG. 19B). Since, like a-MoSₓ, hydrothermal MoS₂ is similarly rich in Mo³⁺-generating sites, and defect-free MoS₂ is low in such sites, it can be concluded that defective sites in Mo sulfides where Mo³⁺—H is generated are the common active sites for both the HER and NAD reduction. Such selective NAD reduction underlines the consistent and specific nature by which Mo sulfide electrocatalysts under HER potentials engage in hydride (as opposed to electron) transfer despite being held at more negative potentials than the NAD dimerization potential. For comparison, GC and Ti catalysts that have been previously reported for specific and direct NADH regeneration were compared to the tested Mo sulfides. However, both were poorly effective under identical conditions, resulting in 4% (GC) and 7% (Ti) conversion of benzaldehyde to benzyl alcohol, respectively, over the course of 3 hours. Furthermore, neither catalyst reached quantitative NADH regeneration under the tested conditions, in contrast to the defective Mo sulfides (see also FIGS. 18A-B).

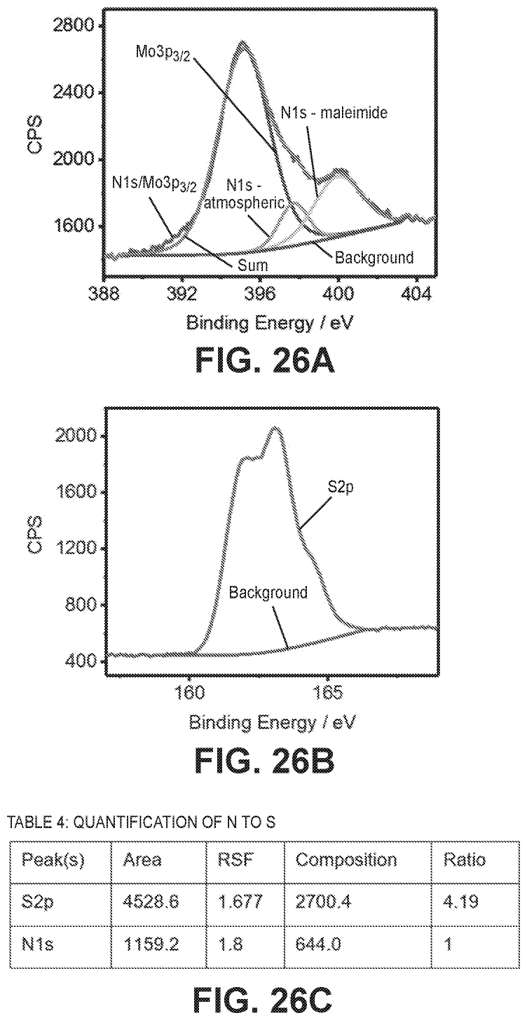

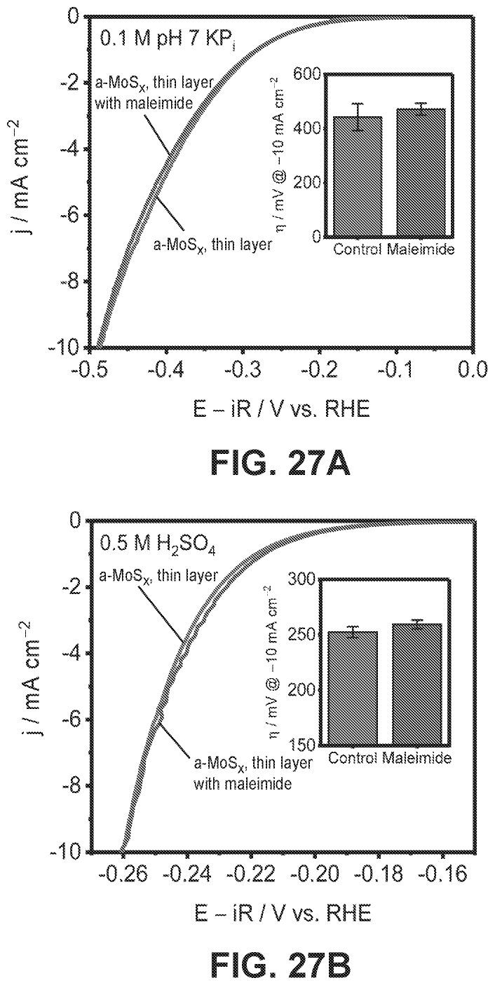

Mo³⁺ hydride formation has implications for both the HER and NAD regeneration. Traditionally, the primary thiol-based model considered that Mo sulfides evolve H₂ through the recombination of two active hydrogen species (H*, hydrogen with a single electron) on S atoms from thiol-like precursors. However, by demonstrating non-dimerization and high 1,4-dihydropyridine yield for both NAD and NMN, it is clear that a hydride must be the intermediate catalytic species. Furthermore, the loss of H* from a thiol would yield a thiyl radical that should have in turn reacted with the reduced NMN to yield decomposition products, as is the case for NADH. These studies demonstrate the viability of a hydride mechanism by testing the HER activity of ultrathin (5 cycle, to minimize the possible contribution of bulk Mo sulfide) a-MoSₓ poisoned by maleimide to inhibit thiol formation. Although x-ray photoelectron spectra (XPS) of the poisoned surfaces suggested that one quarter of all S were poisoned (FIGS. 26A-C (Table 4)), poisoning had no significant effect on HER activity in either proton-rich or proton-poor electrolytes, excluding significant catalytic contributions from thiols regardless of proton concentration (FIGS. 27A and B). It is possible that previous observations of thiols forming during the HER originate from proton accumulation at the cathode as a result of applied potentials. Electronic effects from sulfur also cannot be excluded. Nonetheless, the selective reduction of nicotinamide systems strongly suggests that the Mo³⁺ hydride trapped and observed in EPR is the primary reactive intermediate, thereby allowing Mo sulfides to avoid unwanted dihydropyridine and dimer formation during nicotinamide reduction.

These studies demonstrated that hydride-forming Mo sulfides are economical and selective electrocatalysts for NADH regeneration, in addition to being effective HER catalysts, as a result of the formation of Mo³⁺ hydride active species at cathodic potentials in aqueous solutions. The central role of the hydride species in carrying out both reactions as well as the specificity endowed in nicotinamide reduction provides a convincing picture of the mechanism of reactivity of these hydrides for both reactions. Considering the prevalence of Mo amongst effective non-noble HER catalysts, this study justifies the further design and exploration of Mo-based HER catalysts. Finally, the ability of Mo sulfide electrocatalysts to form and transfer hydrides in exclusion of single electron transfers not only opens a cost-effective route for application in biocatalysis, but points to a new strategy and paradigm for electrocatalyst design.

### Methods

### Materials

Unless otherwise stated, all chemicals were purchased from Sigma-Aldrich and used as received without further purification except Bu₄N PF₆ (98%, Sigma-Aldrich), which was recrystallized twice from boiling ethanol. Purified THE was acquired from a solvent station. Glassy carbon (GC, 0.0707 cm²) and all reference electrodes were acquired from ALS Co., Ltd. Gold wire (99.95%) was acquired from The Nilaco Corporation.

### General Electrochemistry

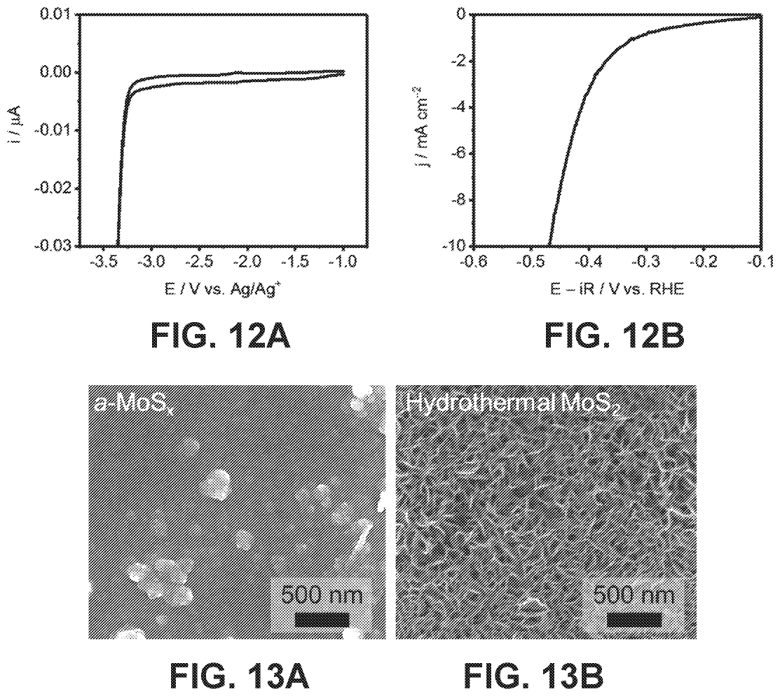

Electrochemical experiments were carried out on a VMP3 Multi-channel or a SP-150 potentiostat (BioLogic). The reference electrode was a Ag/AgCl electrode standardized to the reversible hydrogen electrode (Pt/100% H₂). For organic experiments, the reference electrode was a Ag/Ag⁺ electrode (0.01 M AgNO₃/0.1 M Bu₄N PF₆). The working electrodes were GC, with the exception of the EPR cell in which the working electrode was a flattened (0.3×4 cm) Au wire. The counter electrodes were carbon cloth (when GC was used) or Pt (for organic electrochemistry and EPR). All organic electrochemistry experiments were performed in 0.2 M Bu₄N PF₆ using a custom glass electrochemical cell connected to a Schlenk setup to prevent atmospheric contamination. Linear sweep voltammograms for the correlation plot of Mo³⁺ and HER activity were collected at 1 mV s⁻¹, and the three points were collected using a-MoSₓ deposited at 10, 20, and 40 cycles, in order of the points from lowest to highest activity. (e.g., FIGS. 12A-B).

### Electrodeposition of Mo Sulfides

a-MoSₓ was deposited by cyclic voltammetry for 30 cycles from 2 mM (NH₄)₂MoS₄/0.1 M NaClO₄. Unless otherwise mentioned, the scans were ended on the cathodic edge (i.e., −0.95 V vs. Ag/AgCl). To preserve the trapped hydride, the electrode was quickly washed with degassed water and blow-dried with N2 before being placed in a glovebox antechamber. For samples conditioned by LSV, the electrode was scanned once from open circuit potential (post deposition) to −0.9 V vs. Ag/AgCl in 0.5 M potassium phosphate buffer (pH 7) at 50 mV s⁻¹. Hydrothermal MoS₂ was prepared by autoclave as previously described. Clean GC carbon stubs were placed into the autoclave for the reaction, which was run for 3 hours at 180° C. The crystal pattern was collected using a Bruker D8 Discovery X-ray diffractometer with Cu Kα radiation source. Defect-free MoS₂ was prepared as previously described on a flat GC RDE. (See FIGS. 10-13)

### Maleimide Experiments

A water soluble maleimide derivative (1-hexyl-1H-pyrrole-2,5-dione) was prepared by mixing 1.2 mol of maleic anhydride and 1 mol of 6-aminohexanoic acid in 20 mL of acetic acid and heating at 120° C. for 6 h. Extraction was performed by column chromatography as previously reported. Thin (5 cycle) a-MoSₓ was used in order to minimize the possibility that catalyst in the bulk of the electrode might be active but could not be poisoned. To functionalize maleimide on the surface of a-MoSₓ electrodes, the electrodes were held at −10 mA cm⁻² for 30 seconds and then immediately placed in 10 mM potassium phosphate (pH 7) with 5 mM maleimide derivative for 2 hours. XPS was collected using a Kratos Axis Supra spectrometer with monochromatic Al Kα X-ray source (hv=1486.6 eV) operating at 300 W, multi-channel plate and delay line detector under a vacuum of ˜10⁻⁹ mbar. All spectra were recorded using an aperture slot of 300 μm×700 μm. Survey spectra were collected using a pass energy of 160 eV and a step size of 1 eV. A pass energy of 20 eV and a step size of 0.1 eV were used for the high-resolution spectra. All binding energies were referenced to the C 1s binding energy of 284.8 eV.

### Preparation of N-Methyl Nicotinamide (NMN)

In a fume hood, nicotinamide was heated to reflux in 10 mL CH₃CN with 3 molar equivalents of CH₃I in a 20 mL disposable glass vial with screw cap. The vapor pressure of CH₃CN is sufficiently low that there is no risk of explosion, although the cap can be loosened at reflux to release accumulated pressure. After a few minutes of heating, the white powder turned yellow, indicating the formation of the N-methylated derivative. The reaction was nonetheless left for 1 h, after which the cap was removed and the liquid evaporated (still in the fume hood). The dry powder was confirmed via NMR to be NMN.

### Inductively Coupled Plasma Optical Emission Spectroscopy (ICP-OES)

a-MoSₓ films were dissolved in 1 mL 70% HNO₃. For complete digestion, the sample was then added to 6 mL of 70% HNO₃ and 1 mL 50% HF followed by microwave digestion in a Milestone digestion oven (150 W, 20 min). Afterwards, the samples were diluted by deionized water to 25 mL before being analyzed on an Agilent 5110 ICP-OES spectrometer.

### Electron Paramagnetic Resonance Spectroscopy (EPR)

After deposition, the Au wire electrode was loaded and sealed into a flat quartz cell (Wilmad-LabGlass) and filled with THE in a glovebox. A X-band continuous wave EMX PLUS spectrometer (Bruker, Rheinstetten, Germany), equipped with standard high sensitivity resonator at 9.795 GHz, was used to collect spectra. The spectra were measured at 20 dB microwave attenuation with 5 G modulation amplitude and 100 kHz modulation frequency. For in situ experiments, this setup was modified by using a conductive electrolyte (0.2 M Bu₄N PF₆/THF) with incorporated reference (Ag/Ag⁺) and counter (Pt) electrodes (FIG. 28). The spectrometer was calibrated using a solution of DPPH (g=2.0036). EasySpin (Pepper) was used to model both of the spectra (Mo³⁺—H and Mo³⁺) in order to derive the hyperfine coupling constant of ¹H.

### Electrochemical Reduction of NMN

Reductions were carried out in a 40 mL glass reaction cell at a current density of −10 mA cm⁻². The electrolyte, 0.5 M K₂CO₃/5 mM NMN, was bubbled with Ar throughout the experiment. Control experiments were carried out on bare FTO. UV-vis measurements were carried out using a V-670 spectrophotometer (JASCO). Samples were diluted with deionized water before measurement. For nuclear magnetic resonance spectroscopy (NMR), the reaction was run for four hours, then extracted with ethyl acetate and dried by rotary evaporator under vacuum at room temperature. For deuterated experiments, K₂CO₃ was first dried at 200° C. overnight and kept under anhydrous conditions. D₂O was subsequently used to make the electrolyte. ¹³C, ²H, and ¹H NMR were carried out on 500 and 600 MHz NMR spectrometers (Avance III, Bruker) using CDCl₃ (for ¹³C and ¹H NMR) and CHCl₃ (for ²H NMR). For the starting product, D₂O was used as the solvent.

### Electroenzymatic Conversion of Benzaldehyde to Benzyl Alcohol

In a 4 mL reaction vessel, 2 mL of 0.1 M N-cyclohexyl-2-aminoethanesulfonic acid and 0.1 M K₂SO₄ (pH 9) was used to dissolve 2 μmol of NAD, 20 μmol of benzaldehyde, and 5.6 mg of ADH. a-MoSₓ was deposited on a double-sided GC electrode (exposed area: 2 cm²), which was used to electrolyze the solution at −600 mV vs. RHE. The counter electrode was a glass tube separated from the solution by a Nafion membrane, or a Pt counter electrode was separated from the main compartment by a Nafion membrane with 0.05 M H₂SO₄. Product analysis and quantification was carried out using an Agilent 7890a gas chromatograph/flame ionization detector. A 200 μL aliquot of reaction solution was mixed with 1 mL of ethyl acetate in a small vial; the ethyl acetate was dried and used for analysis. Enzymatic activity for benzaldehyde hydrogenation was confirmed in the same reaction conditions, but with 2 μmol of commercially available NADH. Enzymatic NADH quantitation carried out using Promega (Glo) and Sigma Aldrich kits in both the absence and presence of enzyme and benzaldehyde as described in the text. Standard curves were prepared as provided in the Sigma kit, and using commercial NADH for the Promega kit.

NADH and Dimer Detection with Ultrahigh Performance Liquid Chromatography/Mass Spectrometry

a-MoSₓ was used to reduce a 1 mM solution of NAD in ammonium acetate (0.1 M, pH 9) at −600 mV vs. RHE for 30-60 min. The final solution was diluted with DI water to 1 μg/mL (˜1.5 μM). A Vanquish UHPLC system coupled to an Orbitrap ID-X Tribrid Mass Spectrometer (Thermo Scientific) using positive mode electrospray ionization was used for analysis. The spectrometer was calibrated using the manufacturer's “Calibration Mix ESI” and was confirmed to have high resolution (>120,000) and reliable mass accuracy (<5 ppm). Samples (5 μL each) were infused through a loop injection syringe using a C18 reverse phase column (Agilent, ZORBAX RR Eclipse Plus C18, 2.1×50 mm, 3.5 μm). The eluents were 4 mM dibutylammonium acetate in 95:5 v/v % water/methanol (eluent A) and 25:75 v/v % water/acetonitrile (eluent B). The elution protocol was as previously reported, where eluent B was initially 0% but raised over 8 min to 80%, 100% over 5 min, held at 100% for 3 min and then back to 0% and held for 5 min. The flow rate was 200 μL/min.

### Example 2: Electrochemical Cofactor Regeneration Using Earth Abundant Electrodes for Biocatalytic Applications

Biocatalysis provides a unique and specific pathway towards the formation of otherwise difficult-to-attain compounds, especially in the pharmaceutical industry, due to the specificity and enantioselectivity of enzymatic reactions. Enzymes themselves are divided into seven classes based on the type of reaction that they catalyze. Of these, oxidoreductases (EC class 1) constitute one of the largest classes of enzymes (25% of all known enzymes), and are also of interest for biocatalysis as they catalyze reactions that involve electron transfers. The key to utilizing such enzymes in biocatalytic reactions is the provision of cofactors that serve as electron and proton mediators. In the absence of such mediators, these enzymes cannot carry out any reactions. The most common cofactors that constitute the vast majority of cofactors involved in oxidoreductase reactions are molecules based around flavin and nicotinamide structures, both of which are capable of accepting and holding electrons coupled with protons due to their high energy intermediate structures. The most common cofactors are nicotinamide adenine dinucleotide (NAD⁺ and its reduced form NADH), required by around 80% of all oxidoreductases, as well as its phosphate-added forms (NADP⁺ and NADPH), required by around 10% of all oxidoreductases, and, to a lesser extent, flavin adenine dinucleotide (FAD, and its reduced form FADH₂), also required by around 10% of all oxidoreductases.

Despite the promise of oxidoreductase-based biocatalysis, the provision of reduced cofactors is prohibitively expensive ($3,000 USD/mol NADH, $215,000 USD/mol NADPH). Such an economic limitation is therefore the key barrier to industrial biocatalytic systems, and the cheap and high yield regeneration of cofactors must be demonstrated before any such processes can be commercialized. Consequently, the regeneration of cofactors is an important field of interest in biotechnology. Broadly, studied approaches for cofactor regeneration fall into several categories. Enzymatic approaches seek to mimic biological approaches to cofactor regeneration by harnessing enzymes normally responsible for cofactor regeneration in the natural world. Other chemical approaches involve reducing cofactors with various reducing agents. Photocatalytic approaches rely on photo-generated reducing power from photocatalysts to reduce cofactor, and similarly, electrocatalytic approaches directly utilize electrical current to reduce cofactors. Each of these approaches is a topic of intense study given the potential value of an economical means for cofactor regeneration. In all cases, trade-offs between cost/yield (especially enzymatic, but also chemical) and specificity (photocatalytic, electrocatalytic) mean that limitations remain.

If the specificity challenge can be met, electrochemical approaches show the most theoretical promise as they can provide regeneration at high and controllable rates, with good processing. The problem of specificity arises from the fact that cofactors can be reduced by different mechanisms depending on the role of electrons and hydrides. For example, a one electron reduction of NAD⁺ will lead to the dimerization and irreversible decomposition of NAD. Specific transfer of hydrides (two electrons, one proton) is therefore a necessary requirement for successful and long-lasting electrochemical cofactor regeneration. On many metallic surfaces, electron transfer is far easier than hydride formation and transfer, and so conventional electrochemical approaches have so far proven mostly unsuccessful or rely on good hydride forming metals, mainly noble metals which are not economical for scale-up.

These examples detail use of Group VI compounds (comprising a Group VI element, Cr, Mo, W, and a second non-metal element, namely C, B, P, S, Se, Te, N) as catalysts for cofactor regeneration. These catalysts have several advantages. First, they exclusively form hydrides before they transfer electrons freely as a result of their semiconducting nature. Therefore, they are very specific for cofactor regeneration. Second, although these catalysts can be used with proteins in soluble form, the presence of a second element (the chalcogenide) with defined linker chemistries allows direct protein conjugation to the catalyst without affecting and/or blocking active sites. Finally, these catalysts are composed of earth abundant elements and so their preparation and scale-up is cheap and inexpensive, especially compared to hydride forming catalyst like platinum, gold, or ruthenium-iridium coated titanium. By combining Group VI catalysts in different configurations, it is possible to carry out complicated biocatalytic reactions and cascades in an economically feasible way.

Reduction of N-Methylnicotinamide, an Analog of NAD+, by a-MoSₓ, a Catalyst Deposited on an Electrode.

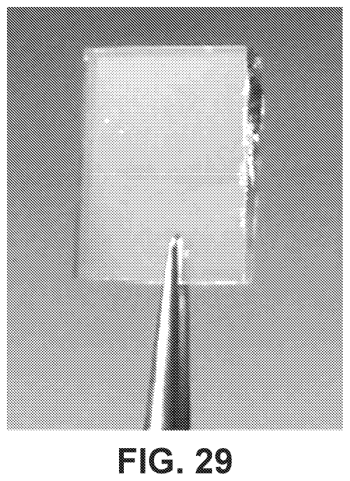

A fluorine-doped tin oxide (FTO) electrode was used for reduction of N-methylnicotinamide. FIG. 29 is a photograph of the a-MoSₓ electrode prepared by electrodeposition for use as a cofactor reduction catalyst. The a-MoSₓ formed a light brown colored layer on the top half of the electrode, and the white colored layer on the bottom half of the electrode which is being held with tweezers is uncolored FTO. The he FTO control sample had no catalyst, and otherwise functioned as a conductor, as shown in FIG. 7C. A peak at 360 nm corresponds to the presence of hydride-reduced N-methylnicotinamide, the equivalent reaction for NAD⁺/NADP⁺→NADH/NADPH. When the FTO is used to carry out the same reaction as a-MoSₓ (inset), it is not only less active for forming the hydride-reduced product, FTO also forms a breakdown product at 300 nm. Therefore, a-MoSₓ is necessary for proper cofactor reduction.

### Reduction of NAD+ for Use by Immobilized Alcohol Dehydrogenase (ADH).

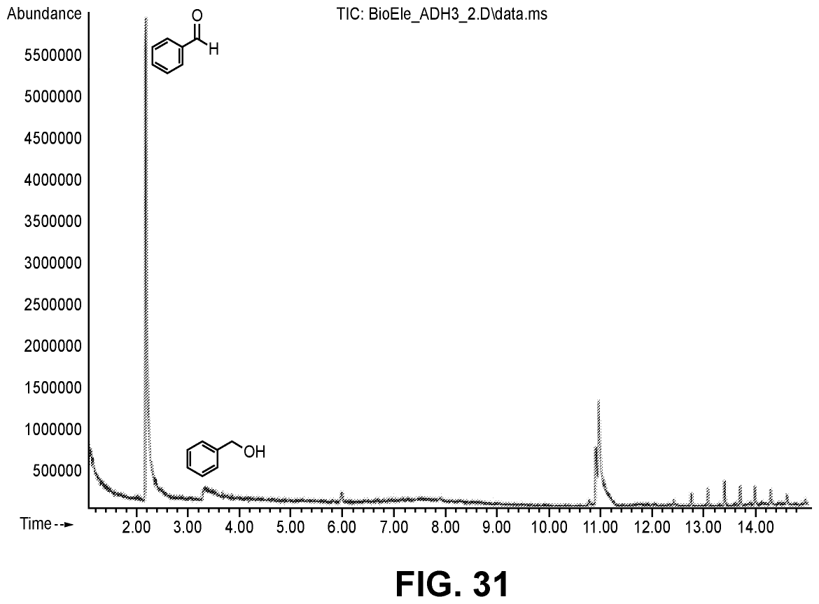

FIG. 31 shows the reduction potential of relevant electrochemical reduction half-reactions. The three major oxidoreductase cofactors are included, as is the formation potential (estimated) for a Group VI metal hydride, where “M” is the Group VI metal.

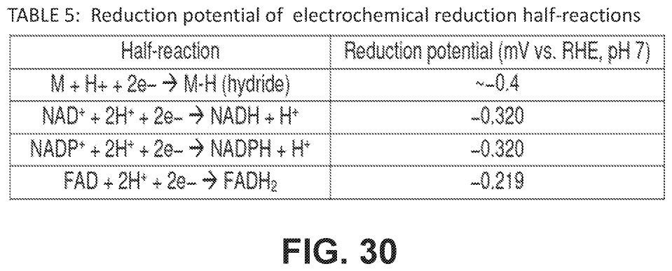

As shown in FIGS. 17-19, reduction of benzaldehyde to benzyl alcohol by alcohol dehydrogenase (ADH), an oxidoreductase that utilizes NADH as a cofactor, which in turn was reduced by a-MoSₓ. The reaction was carried out 0.2 V more negative than the minimum formation potential of the hydride (FIG. 30), resulting in a yield of 78% over the course of 3 h.

ADH was immobilized on the surface of the electrode, operated in electrolyte with NAD+ and benzaldehyde. After 1 h, 10% of the benzaldehyde was converted to benzyl alcohol as determined by GC-MS (FIG. 31)

The Examples above should not be construed as limiting the scope of the disclosure, but as merely providing illustrations of some of the embodiments of this disclosure. The example embodiments, as described above, were chosen and described in order to best explain the principles of the disclosure and its practical application to thereby enable others skilled in the art to best utilize the disclosure in various embodiments and with various modifications as are suited to the particular use contemplated. Various combinations or sub-combinations of specific features and aspects of the embodiments fall within the scope of this disclosure. Various features and aspects of the disclosed embodiments can be combined with or substituted for one another. The scope of the disclosure is defined by the claims appended hereto.

1. A method of electrochemical cofactor regeneration comprising:
holding an electrode comprising a Group VI transition metal chalcogenide catalyst at a potential sufficient to form a metal hydride in an aqueous electrolyte solution;
contacting the electrode with an oxidized cofactor to reduce the cofactor.

2. The method of claim 1, wherein the oxidized cofactor is selected from the group consisting of cofactor of NAD⁺, NADP⁺, FAD⁺ and FMN⁺ or a combination thereof.

3. The method of claim 1, wherein the potential is held within the range of about −0.3V to −0.6 V.

4. The method of claim 1, wherein the Group VI transition metal chalcogenide catalyst has the formula MEₓ, where M is a Group VI transition metal, E is a non-metal element, and x is a number greater than 2.

5. The method of claim 4, wherein M is selected from Cr, Mo, and W and/or E is selected from the group of non-metal elements consisting of B, C, N, S, Se, Te, and P.

6. The method of claim 1, wherein the Group VI transition metal chalcogenide catalyst is selected from the group of MoSₓ, MoSeₓ, WSeₓ, and WSₓ, wherein x is a number greater than 2.

7. The method of claim 1, wherein the Group VI transition metal chalcogenide catalyst is amorphous.

8. (canceled)

9. The method of claim 1, wherein the aqueous electrolyte solution comprises at least one of potassium phosphate, sodium phosphate and potassium perchlorate.

10. A method of improving the rate of an oxidoreductase-catalyzed reaction comprising:
reacting an oxidoreductase and a substrate thereof in the presence of an oxidoreductase cofactor, whereby the substrate is converted to a first product and the cofactor is oxidized;
regenerating the oxidized cofactor with an electrode comprising a Group VI transition metal chalcogenide catalyst at a potential sufficient to form a metal hydride,
wherein the rate of the reaction is improved compared to the rate of a corresponding oxidoreductase-catalyzed reaction performed without regenerating the oxidized cofactor.

11. The method of claim 10, wherein the oxidoreductase is selected from the group consisting of nicotinamide-dependent oxidoreductases, NADH-dependent oxidoreductases, NADPH-dependent oxidoreductases, and FADH₂-dependent oxidoreductases.

12. The method of claim 11, wherein the oxidoreductase is selected from the group consisting of alcohol dehydrogenases, aldehyde dehydrogenases, ene reductases, amino acid dehydrogenases, oxidoreductases of CH—NH groups, nitrate reductases, oxidoreductases acting on a sulfur group, dehydrogenases of diphenols, peroxidases, hydrogenases, oxygenases, monooxygenases, oxidoreductases of metal ions, oxidoreductases acting on CH or CH₂ groups, oxidoreductases of iron-sulfur proteins and of flavodoxin, reductive dehalogenases, and oxidoreductases reducing a C—O—C group.

13. The method of claim 10, wherein the oxidoreductase is conjugated to the electrode.

14. The method of claim 10, wherein the oxidoreductase is solubilized in the aqueous electrolyte solution.

15. The method of claim 10, wherein the electrode is contained in a reaction vessel and the oxidoreductase is separated from the electrode by a membrane.

16. (canceled)

17. The method of claim 10, further comprising a step of:
reacting a second enzyme and the first product, whereby the first product is converted to a second product.

18. (canceled)

19. A system for an oxidoreductase-catalyzed reaction comprising:
a bioreactor comprising a reaction vessel;
wherein the reaction vessel is configured to contain an electrode comprising a Group VI transition metal chalcogenide catalyst and an aqueous electrolyte solution; and configured for regenerating an oxidoreductase cofactor;
wherein the bioreactor is configured for reacting the oxidoreductase and a substrate thereof in the presence of the regenerated cofactor, and wherein regenerating the oxidoreductase cofactor includes holding the electrode comprising the Group VI transition metal chalcogenide catalyst at a potential sufficient to form a metal hydride in an aqueous electrolyte solution and contacting the electrode with an oxidized cofactor to reduce the cofactor.

20. The system of claim 19, further comprising a membrane configured to separate the oxidoreductase and the electrode.

21. The system of claim 20, wherein the membrane is a dialysis membrane.

22. The system of claim 20, wherein the oxidoreductase is immobilized on the membrane.

23. The system of claim 19, wherein the oxidoreductase is conjugated to the electrode.

24. (canceled)
